(19) United States
(12) Patent Application Publication
(10) Pub. No.: US 2022/0056511 A1
(43) Pub. Date: Feb. 24, 2022

(54) METHODS AND COMPOSITIONS TO REDUCE NONSPECIFIC AMPLIFICATION IN ISOTHERMAL AMPLIFICATION REACTIONS

(71) Applicant: Alveo Technologies, Inc., Alameda, CA (US)

(72) Inventors: Joseph Carl Gaiteri, Emeryville, CA (US); Rixun Fang, Menlo Park, CA (US); Brenna Hearn Lord, San Francisco, CA (US); Yuh-Min Chiang, Sunnyvale, CA (US); Ronald Phillip Chiarello, Orinda, CA (US)

(21) Appl. No.: 17/416,105

(22) PCT Filed: Dec. 18, 2019
(86) PCT No.: PCT/US2019/067134

§ 371 Date: Jun. 18, 2021

Related U.S. Application Data
(60) Provisional application No. 62/782,610, filed on Dec. 20, 2018.

(51) Int. Cl. C12Q 1/6848; C12Q 1/6876; C12N 9/12

(52) U.S. Cl. CPC C12Q 1/6848; C12Y 207/07007; C12N 9/1252; C12Q 1/6876

(57) ABSTRACT

Embodiments relate to methods, systems and compositions for reducing nonspecific amplification in isothermal amplification reactions. Some embodiments relate to reducing nonspecific amplification in loop-mediated isothermal amplification (LAMP) reactions with certain oligonucleotides.

### RELATED APPLICATIONS

This application claims priority to U.S. Prov. App. 62/782,610 filed Dec. 20, 2018 entitled “METHODS AND COMPOSITIONS TO REDUCE NONSPECIFIC AMPLIFICATION IN ISOTHERMAL AMPLIFICATION REACTIONS” which is expressly incorporated by reference in its entirety.

### REFERENCE TO SEQUENCE LISTING

The present application is being filed along with a Sequence Listing in electronic format. The Sequence Listing is provided as a file entitled ALVEO016WOSEQLISTING, created Dec. 7, 2019, which is approximately 31 Kb in size. The information in the electronic format of the Sequence Listing is incorporated herein by reference in its entirety.

### FIELD OF THE INVENTION

Embodiments relate to methods, systems and compositions for reducing nonspecific amplification or otherwise improving isothermal amplification reactions. Some embodiments relate to reducing nonspecific amplification in loop-mediated isothermal amplification (LAMP) reactions using certain oligonucleotides.

### BACKGROUND OF THE INVENTION

Since being developed in 1983, the polymerase chain reaction (PCR) has played a central role in nucleic acid amplification. However, the PCR assay requires an expensive thermal cycler to amplify the DNA fragment in multiple temperature-dependent steps.

The loop-mediated isothermal amplification (LAMP) assay is another nucleic acid amplification technique. In contrast to PCR, the LAMP assay can amplify a targeted sequence at a constant temperature. Therefore, a large and costly thermal cycler is not necessary for a LAMP assay. The LAMP assay uses a single DNA polymerase with strong strand displacement activity and a set of 4-6 specially designed primers facilitating rapid isothermal amplification (typically at 60-70° C.) of a DNA or RNA nucleic acid target. Positive results can be identified visually by turbidity or addition of fluorescent DNA-binding dyes. However, LAMP assays are often prone to the appearance of false positive results. See e.g., Senarath, K. D., et al., Journal of Tuberculosis Research, 2014, 2, 168-172; Nagai K., et al., Sci. Rep. 6, 39090; doi: 10.1038/srep39090 2016; and Suleman E., et al., J Vet Diagn Invest. 2016 September; 28(5):536-42. Thus, there is a need for more robust LAMP assays.

### SUMMARY OF THE INVENTION

Some embodiments of the methods and compositions provided herein include an aqueous solution comprising: a set of loop-mediated isothermal amplification (LAMP) primers sufficient to perform a LAMP reaction of a target nucleic acid; a polymerase; and a first inhibitor oligonucleotide comprising a hairpin, wherein: the first inhibitor oligonucleotide does not specifically hybridize to the target nucleic acid, and the first inhibitor oligonucleotide has activity to reduce the level of a nonspecific amplification product of the LAMP reaction compared to the level of a nonspecific amplification product of a LAMP reaction performed in the absence of the first inhibitor oligonucleotide.

In some embodiments, the 3′ end of the first inhibitor oligonucleotide comprises a blocking moiety which inhibits polymerase extension of the first inhibitor oligonucleotide. In some embodiments, the blocking moiety is selected from a phosphate, a C3 spacer, an amine, biotin, or an inverted base. In some embodiments, the 3′ end of the first inhibitor oligonucleotide is phosphorylated.

In some embodiments, the first inhibitor oligonucleotide lacks a nucleotide comprising uracil or inosine.

In some embodiments, the hairpin has a Tₘ less than about 65° C. In some embodiments, the hairpin has a Tₘ less than about 55° C.

In some embodiments, a 3′ terminal nucleotide of the first inhibitor oligonucleotide is single-stranded, and a nucleotide of the first inhibitor oligonucleotide consecutive with the 3′ terminal nucleotide is double-stranded.

In some embodiments, the hairpin comprises a loop comprising or consisting of three consecutive single-stranded nucleotides.

In some embodiments, the first inhibitor oligonucleotide comprises, consists of, or consists essentially of: a nucleic acid having at least 90% sequence identity with the nucleotide sequence selected from any one of SEQ ID NOs:01-15; a nucleic acid capable of hybridizing or configured to hybridize to the complement of a nucleic acid having the nucleotide sequence selected from any one of SEQ ID NOs:01-15; or a nucleic acid having the nucleotide sequence selected from any one of SEQ ID NOs:01-15.

In some embodiments, the first inhibitor oligonucleotide comprises, consists of, or consists essentially of: a nucleic acid having at least 90% sequence identity with the nucleotide sequence of SEQ ID NO:09; a nucleic acid capable of hybridizing or configured to hybridize to the complement of a nucleic acid having the nucleotide sequence of SEQ ID NO:09; or a nucleic acid having the nucleotide sequence of SEQ ID NO:09.

In some embodiments, the first inhibitor oligonucleotide comprises, consists of, or consists essentially of: a nucleic acid having at least 90% sequence identity with the nucleotide sequence of SEQ ID NO:01; a nucleic acid capable of hybridizing or configured to hybridize to the complement of a nucleic acid having the nucleotide sequence of SEQ ID NO:01; or a nucleic acid having the nucleotide sequence of SEQ ID NO:01.

In some embodiments, the first inhibitor oligonucleotide comprises, consists of, or consists essentially of: a nucleic acid having at least 90% sequence identity with the nucleotide sequence of SEQ ID NO:02; a nucleic acid capable of hybridizing or configured to hybridize to the complement of a nucleic acid having the nucleotide sequence of SEQ ID NO:02; or a nucleic acid having the nucleotide sequence of SEQ ID NO:02.

In some embodiments, the LAMP reagent mix further comprises a second inhibitor oligonucleotide. In some embodiments, the 3′ end of the second inhibitor oligonucleotide comprises a blocking moiety which inhibits polymerase extension of the second inhibitor oligonucleotide. In some embodiments, the 3′ end of the second inhibitor oligonucleotide is phosphorylated.

In some embodiments, a 3′ terminal nucleotide of the second inhibitor oligonucleotide is single-stranded, and a nucleotide of the second inhibitor oligonucleotide consecutive with the 3′ terminal nucleotide is double-stranded.

In some embodiments, a ratio between the first inhibit oligonucleotide and the second inhibitor oligonucleotide in the aqueous solution is in a range between 1:10 and 1:1. In some embodiments, the ratio is about 1:5 or 1:5.

In some embodiments, the second inhibitor oligonucleotide comprises, consists of, or consists essentially of: a nucleic acid having at least 90% sequence identity with the nucleotide sequence selected from any one of SEQ ID NOs:01-15; a nucleic acid capable of hybridizing or configured to hybridize to the complement of a nucleic acid having a nucleotide sequence selected from any one of SEQ ID NOs:01-15; or a nucleic acid having the nucleotide sequence selected from any one of SEQ ID NOs:01-15.

In some embodiments, the second inhibitor oligonucleotide comprises, consists of, or consists essentially of: a nucleic acid having at least 90% sequence identity with the nucleotide sequence of SEQ ID NO:09; a nucleic acid capable of hybridizing or configured to hybridize to the complement of a nucleic acid having a nucleotide sequence of SEQ ID NO:09; or a nucleic acid having the nucleotide sequence of SEQ ID NO:09.

Some embodiments also include a crowding agent. In some embodiments, the crowding agent is selected from polyethylene glycol (PEG), dextran, polyvinyl alcohol, polyvinyl pyrrolidone, or Ficoll. In some embodiments, the crowding agent is selected from PEG-35K, PEG-8K or Ficoll-400K. In some embodiments, the crowding agent comprises PEG-35K.

In some embodiments, the polymerase comprises a strand displacing activity. In some embodiments, the polymerase is selected from Bst large fragment, Bca (exo-), Vent, Vent (exo-), Deep Vent, Deep Vent (exo-), phi29 phage, MS-2 phage, Taq, Z-Taq, KOD, Klenow fragment, Bst 2.0, Bst 3.0, a Bst derivative, a Bsu polymerase, a Gsp polymerase, a Sau polymerase or any combination thereof. In some embodiments, the polymerase comprises a Bst large fragment.

In some embodiments, the first inhibitor oligonucleotide has a concentration in a range from 0.1 μM to 20 μM or about 0.1 μM to about 20 μM.

Some embodiments also include a plurality of different sets of LAMP primers, each set sufficient to perform a LAMP reaction of a different target nucleic acid. In some embodiments, a primer of the set of LAMP primers comprises the nucleotide sequence selected from any one of SEQ ID NOs:19-162. In some embodiments, the set of LAMP primers comprises a FIP primer and a BIP primer, each primer having the nucleotide sequence selected from any one of SEQ ID NOs:19-162. In some embodiments, the set of LAMP primers comprises a F3 primer, a B3 primer, a FIP primer, a BIP primer, a LF primer and a LB primer, each primer having the nucleotide sequence selected from any one of SEQ ID NOs:19-162.

In some embodiments, the target nucleic acid is a nucleic acid from a virus or organism selected from Dengue virus, Influenza A virus strain H3N1, Influenza A virus strain H3N2, Haemophilus influenzae, Hepatitis A virus, Hepatitis B virus, Hepatitis C virus, Human immunodeficiency virus-1, Plasmodium spp, Bacteriophage MS2, Parvovirus B19, Respiratory syncytial virus, Salmonella typhimurium, strain LT2, Mycobacterium tuberculosis, or Zika virus.

In some embodiments, the first inhibitor oligonucleotide has activity to increase a critical time (Ct) value for the amplification of a false positive in the LAMP reaction compared to a Ct value for the amplification of the false positive in a LAMP reaction performed in the absence of the first inhibitor oligonucleotide. In some embodiments, the increase is at least 2-fold. In some embodiments, the increase is at least 3-fold. In some embodiments, the increase is at least 10 minutes. In some embodiments, the increase is at least 15 minutes.

Some embodiments of the methods and compositions provided herein include a method of reducing nonspecific amplification in a loop-mediated isothermal amplification (LAMP) reaction with a target nucleic acid, comprising: providing a LAMP reagent mix comprising the aqueous solution of any one of the foregoing aqueous solutions; and performing the LAMP reaction with the LAMP reagent mix in the presence of the target nucleic acid, wherein the level of a nonspecific amplification product of the LAMP reaction is reduced compared to the level of a nonspecific amplification product of a LAMP reaction performed in the absence of the first inhibitor oligonucleotide.

In some embodiments, a critical time (Ct) value for the amplification of a false positive in the LAMP reaction is increased compared to a Ct value for the amplification of the false positive in a LAMP reaction performed in the absence of the first inhibitor oligonucleotide. In some embodiments, the increase in the Ct value is at least 2-fold. In some embodiments, the increase in the Ct value is at least 3-fold. In some embodiments, the increase in the Ct value is at 10 minutes. In some embodiments, the increase in the Ct value is at 15 minutes.

In some embodiments, an amplification product of the LAMP reaction is detected by changes in a signal selected from an optical signal, a pH signal, and an electrical signal. In some embodiments, an amplification product of the LAMP reaction is detected by changes in an electrical signal.

Some embodiments of the methods and compositions provided herein include an isolated inhibitor oligonucleotide comprising a hairpin, wherein the inhibitor oligonucleotide has activity to reduce the level of a nonspecific amplification product of a LAMP reaction compared to the level of a nonspecific amplification product of a LAMP reaction performed in the absence of the inhibitor oligonucleotide.

In some embodiments, the 3′ end of the inhibitor oligonucleotide comprises a blocking moiety which inhibits polymerase extension of the first inhibitor oligonucleotide. In some embodiments, the blocking moiety is selected from a phosphate, a C3 spacer, an amine, biotin, or an inverted base. In some embodiments, the 3′ end of the inhibitor oligonucleotide is phosphorylated.

In some embodiments, the first inhibitor oligonucleotide lacks a nucleotide comprising uracil or inosine.

In some embodiments, the hairpin has a Tₘ less than about 65° C. In some embodiments, the hairpin has a Tₘ less than about 55° C.

In some embodiments, a 3′ terminal nucleotide of the inhibitor oligonucleotide is single-stranded, and a nucleotide of the inhibitor oligonucleotide consecutive with the 3′ terminal nucleotide is double-stranded. In some embodiments, the hairpin comprises a loop comprising or consisting of three consecutive single-stranded nucleotides.

In some embodiments, the inhibitor oligonucleotide comprises, consists of, or consists essentially of: a nucleic acid having at least 90% sequence identity with the nucleotide sequence selected from any one of SEQ ID NOs:01-15; a nucleic acid capable of hybridizing or configured to hybridize to the complement of a nucleic acid having the nucleotide sequence selected from any one of SEQ ID NOs:01-15; or a nucleic acid having the nucleotide sequence selected from any one of SEQ ID NOs:01-15.

In some embodiments, the inhibitor oligonucleotide comprises, consists of, or consists essentially of: a nucleic acid having at least 90% sequence identity with the nucleotide sequence of SEQ ID NO:09; a nucleic acid capable of hybridizing or configured to hybridize to the complement of a nucleic acid having the nucleotide sequence of SEQ ID NO:09; or a nucleic acid having the nucleotide sequence of SEQ ID NO:09.

In some embodiments, the inhibitor oligonucleotide comprises, consists of, or consists essentially of: a nucleic acid having at least 90% sequence identity with the nucleotide sequence of SEQ ID NO:01; a nucleic acid capable of hybridizing or configured to hybridize to the complement of a nucleic acid having the nucleotide sequence of SEQ ID NO:01; or a nucleic acid having the nucleotide sequence of SEQ ID NO:01.

In some embodiments, the inhibitor oligonucleotide comprises, consists of, or consists essentially of: a nucleic acid having at least 90% sequence identity with the nucleotide sequence of SEQ ID NO:02; a nucleic acid capable of hybridizing or configured to hybridize to the complement of a nucleic acid having the nucleotide sequence of SEQ ID NO:02; or a nucleic acid having the nucleotide sequence of SEQ ID NO:02.

Some embodiments of the methods and compositions provided herein include a kit comprising: a first inhibitor oligonucleotide comprising the inhibitor oligonucleotide of any one of the foregoing inhibitor oligonucleotides; and a reagent selected from: a polymerase comprising a strand displacement activity, or a set of loop-mediated isothermal amplification (LAMP) primers sufficient to perform a LAMP reaction of a target nucleic acid.

Some embodiments also include a second inhibitor oligonucleotide. In some embodiments, the 3′ end of the second inhibitor oligonucleotide comprises a blocking moiety which inhibits polymerase extension of the second inhibitor oligonucleotide. In some embodiments, the 3′ end of the second inhibitor oligonucleotide is phosphorylated. In some embodiments, a 3′ terminal nucleotide of the second inhibitor oligonucleotide is single-stranded, and a nucleotide of the second inhibitor oligonucleotide consecutive with the 3′ terminal nucleotide is double-stranded.

In some embodiments, a ratio between the first inhibit oligonucleotide and the second inhibitor oligonucleotide in the aqueous solution is in a range between 1:10 and 1:1. In some embodiments, the ratio is about 1:5 or 1:5.

In some embodiments, the second inhibitor oligonucleotide comprises, consists of, or consists essentially of: a nucleic acid having at least 90% sequence identity with the nucleotide sequence selected from any one of SEQ ID NOs:01-15; a nucleic acid capable of hybridizing or configured to hybridize to the complement of a nucleic acid having a nucleotide sequence selected from any one of SEQ ID NOs:01-15; or a nucleic acid having the nucleotide sequence selected from any one of SEQ ID NOs:01-15.

In some embodiments, the second inhibitor oligonucleotide comprises, consists of, or consists essentially of: a nucleic acid having at least 90% sequence identity with the nucleotide sequence of SEQ ID NO:09; a nucleic acid capable of hybridizing or configured to hybridize to the complement of a nucleic acid having a nucleotide sequence of SEQ ID NO:09; or a nucleic acid having the nucleotide sequence of SEQ ID NO:09.

Some embodiments also include a plurality of different sets of LAMP primers, each set sufficient to perform a LAMP reaction of a different target nucleic acid. In some embodiments, the set of LAMP primers comprises at least 4 different primers. In some embodiments, the set of LAMP primers comprises at least 6 different primers. In some embodiments, a primer of the set of LAMP primers comprises the nucleic acid sequence of any one of SEQ ID NOs:19-162. In some embodiments, the set of LAMP primers comprises a FIP primer, and a BIP primer, each primer having the nucleic acid sequence of any one of SEQ ID NOs:19-162. In some embodiments, the set of LAMP primers comprises a F3 primer, a B3 primer, a FIP primer, a BIP primer, a LF primer and a LB primer, each primer having the nucleic acid sequence of any one of SEQ ID NOs:19-162.

In some embodiments, the target nucleic acid is a nucleic acid from a virus or organism selected from Dengue virus, Influenza A virus strain H3N1, Influenza A virus strain H3N2, Haemophilus influenzae, Hepatitis A virus, Hepatitis B virus, Hepatitis C virus, Human immunodeficiency virus-1, Plasmodium spp, Bacteriophage MS2, Parvovirus B19, Respiratory syncytial virus, Salmonella typhimurium, strain LT2, Mycobacterium tuberculosis, or Zika virus.

In some embodiments, the polymerase is selected from Bst large fragment, Bca (exo-), Vent, Vent (exo-), Deep Vent, Deep Vent (exo-), phi29 phage, MS-2 phage, Taq, Z-Taq, KOD, Klenow fragment, Bst 2.0 (NEB), Bst 3.0 (NEB), a Bst derivative, a Bsu polymerase, a Gsp polymerase, a Sau polymerase or any combination thereof. In some embodiments, the polymerase comprises a Bst large fragment.

In some embodiments, the reagent mix comprises a crowding agent. In some embodiments, the crowding agent is selected from polyethylene glycol (PEG), dextran, polyvinyl alcohol, polyvinyl pyrrolidone, or Ficoll. In some embodiments, the crowding agent is selected from PEG-35K, PEG-8K or Ficoll-400K. In some embodiments, the crowding agent comprises PEG-35K.

Some embodiments of the methods and compositions provided herein include a system for detecting a target nucleic acid in a loop-mediated isothermal amplification (LAMP) reaction, comprising a vessel comprising the aqueous solution of any one of foregoing aqueous solutions; and a detector configured to detect an amplification product in the vessel. Some embodiments also include the target nucleic acid. In some embodiments, the detector is configured to detect a change in an electrical signal or an optical signal. In some embodiments, the detector is configured to detect a change in an electrical signal.

### BRIEF DESCRIPTION OF THE DRAWINGS

FIG. 1A depicts a predicted secondary structure for a HAVFIP1 oligonucleotide (SEQ ID NO:01).

FIG. 1B depicts a predicted secondary structure for an extended HAVFIP1 oligonucleotide (SEQ ID NO:08).

FIG. 2 depicts predicted secondary structures for various oligonucleotides including SEQ ID NO:01, SEQ ID NO:01, SEQ ID NO:94, SEQ ID NO:15, SEQ ID NO:11, SEQ ID NO:17, SEQ ID NO:13, SEQ ID NO:16, SEQ ID NO:107, SEQ ID NO:03, SEQ ID NO:04, SEQ ID NO:05, and SEQ ID NO:10.

FIG. 3 is a graph summarizing critical time (Ct) values for different concentrations of target for reactions containing different crowding agents.

### DETAILED DESCRIPTION

Embodiments relate to methods, systems and compositions for reducing nonspecific amplification or otherwise improving isothermal amplification reactions. Some embodiments relate to reducing nonspecific amplification or otherwise improving loop-mediated isothermal amplification (LAMP) reactions using certain oligonucleotides. In some embodiments, certain inhibitor oligonucleotides have activity to reduce nonspecific amplification in a LAMP reaction. For example, in certain LAMP reactions, the presence of an inhibitor oligonucleotide can suppress the amplification of non-target nucleic acids. In some such embodiments, the amplification of non-target nucleic acids in a LAMP reaction in the presence of an inhibitor oligonucleotide is detected at a substantially higher critical time (Ct) value, compared to detection of amplification of non-target nucleic acids in a reaction performed in the absence of an inhibitor oligonucleotide. In some embodiments, the presence of an inhibitor oligonucleotide inhibits amplification of non-target nucleic acids. Some embodiments provided herein include embodiments disclosed in Int. App. Pub. No. WO 2016/057422, U.S. 2016/0097740, U.S. 2016/0097741, U.S. 2016/0097739, U.S. 2016/0097742, U.S. 2016/0130639; and Int. App. Pub. No. WO 2018/057647 which claims priority to U.S. App No. 62/398,959, U.S. App No. 62/399,047, U.S. App No. 62/398,925, U.S. App No. 62/398,913, U.S. App No. 62/398,955, or U.S. App No. 62/398,965, which are each incorporated by reference in its entirety. Some embodiments provided herein include embodiments disclosed in: U.S. 62/783,117 filed on Dec. 20, 2018 entitled “ISOTHERMAL AMPLIFICATION WITH ELECTRICAL DETECTION”; U.S. 62/783,104 filed on Dec. 20, 2018 entitled “HANDHELD IMPEDANCE-BASED DIAGNOSTIC TEST SYSTEM FOR DETECTING ANALYTES”; or U.S. 62/783,051 filed on Dec. 20, 2018 entitled “METHODS AND COMPOSITIONS FOR DETECTION OF AMPLIFICATION PRODUCTS”, the entire contents of which are each expressly incorporated by reference in its entirety.

### Definitions

As used herein the term “nucleic acid” and/or “oligonucleotide” and/or grammatical equivalents thereof can refer to at least two nucleotide monomers linked together. A nucleic acid can generally contain phosphodiester bonds; however, in some embodiments, nucleic acid analogs may have other types of backbones, comprising, for example, phosphoramide (Beaucage, et al., Tetrahedron, 49:1925 (1993); Letsinger, J. Org. Chem., 35:3800 (1970); Sprinzl, et al., Eur. J. Biochem., 81:579 (1977); Letsinger, et al., Nucl. Acids Res., 14:3487 (1986); Sawai, et al., Chem. Lett., 805 (1984), Letsinger, et al., J. Am. Chem. Soc., 110:4470 (1988); and Pauwels, et al., Chemica Scripta, 26:141 (1986)), phosphorothioate (Mag, et al., Nucleic Acids Res., 19:1437 (1991); and U.S. Pat. No. 5,644,048), phosphorodithioate (Briu, et al., J. Am. Chem. Soc., 111:2321 (1989), O-methylphosphoroamidite linkages (see Eckstein, Oligonucleotides and Analogues: A Practical Approach, Oxford University Press), and peptide nucleic acid backbones and linkages (see Egholm, J. Am. Chem. Soc., 114:1895 (1992); Meier, et al., Chem. Int. Ed. Engl., 31:1008 (1992); Nielsen, Nature, 365:566 (1993); Carlsson, et al., Nature, 380:207 (1996)).

Other analog nucleic acids include those with positive backbones (Denpcy, et al., Proc. Natl. Acad. Sci. USA, 92:6097 (1995)); non-ionic backbones (U.S. Pat. Nos. 5,386,023; 5,637,684; 5,602,240; 5,216,141; and 4,469,863; Kiedrowshi, et al., Angew. Chem. Intl. Ed. English, 30:423 (1991); Letsinger, et al., J. Am. Chem. Soc., 110:4470 (1988); Letsinger, et al., Nucleosides & Nucleotides, 13:1597 (1994); Chapters 2 and 3, ASC Symposium Series 580, “Carbohydrate Modifications in Antisense Research”, Ed. Y. S. Sanghui and P. Dan Cook; Mesmaeker, et al., Bioorganic & Medicinal Chem. Lett., 4:395 (1994); Jeffs, et al., J. Biomolecular NMR, 34:17 (1994); Tetrahedron Lett., 37:743 (1996)) and non-ribose (U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Coo). Nucleic acids may also contain one or more carbocyclic sugars (see Jenkins, et al., Chem. Soc. Rev., (1995) pp. 169 176).

Modifications of the ribose-phosphate backbone may be done to facilitate the addition of additional moieties such as labels, or to increase the stability of such molecules under certain conditions. In addition, mixtures of naturally occurring nucleic acids and analogs can be made. Alternatively, mixtures of different nucleic acid analogs, and mixtures of naturally occurring nucleic acids and analogs may be made. The nucleic acids may be single stranded or double stranded, as specified, or contain portions of both double stranded or single stranded sequence. The nucleic acid may be DNA, for example, genomic or cDNA, RNA or a hybrid, from single cells, multiple cells, or from multiple species, as with metagenomic samples, such as from environmental samples. A nucleic acid can contain any combination of deoxyribo- and ribo-nucleotides, and any combination of bases, including uracil, adenine, thymine, cytosine, guanine, inosine, xanthanine, hypoxanthanine, isocytosine, isoguanine, or base analogs such as nitropyrrole (including 3-nitropyrrole) or nitroindole (including 5-nitroindole), etc.

In some embodiments, a nucleic acid can include at least one promiscuous base. Promiscuous bases can base-pair with more than one different type of base. In some embodiments, a promiscuous base can base-pair with at least two different types of bases and no more than three different types of bases. An example of a promiscuous base includes inosine that may pair with adenine, thymine, or cytosine. Other examples include hypoxanthine, 5-nitroindole, acylic 5-nitroindole, 4-nitropyrazole, 4-nitroimidazole or 3-nitropyrrole (Loakes et al., Nucleic Acid Res. 22:4039 (1994); Van Aerschot et al., Nucleic Acid Res. 23:4363 (1995); Nichols et al., Nature 369:492 (1994); Bergstrom et al., Nucleic Acid Res. 25:1935 (1997); Loakes et al., Nucleic Acid Res. 23:2361 (1995); Loakes et al., J. Mol. Biol. 270:426 (1997); and Fotin et al., Nucleic Acid Res. 26:1515 (1998)). Promiscuous bases that can base-pair with at least three, four or more types of bases can also be used.

As used herein, the term “nucleotide analog” and/or grammatical equivalents thereof can refer to synthetic analogs having modified nucleotide base portions, modified pentose portions, and/or modified phosphate portions, and, in the case of polynucleotides, modified internucleotide linkages, as generally described elsewhere (e.g., Scheit, Nucleotide Analogs, John Wiley, New York, 1980; Englisch, Angew. Chem. Int. Ed. Engl. 30:613-29, 1991; Agarwal, Protocols for Polynucleotides and Analogs, Humana Press, 1994; and S. Verma and F. Eckstein, Ann. Rev. Biochem. 67:99-134, 1998). Generally, modified phosphate portions comprise analogs of phosphate wherein the phosphorous atom is in the +5 oxidation state and one or more of the oxygen atoms is replaced with a non-oxygen moiety, e.g., sulfur. Exemplary phosphate analogs include but are not limited to phosphorothioate, phosphorodithioate, phosphoroselenoate, phosphorodiselenoate, phosphoroanilothioate, phosphoranilidate, phosphoramidate, or boronophosphates, including associated counterions, e.g., H+, NH₄+, Na+, if such counterions are present. Example modified nucleotide base portions include but are not limited to 5-methylcytosine (5mC); C-5-propynyl analogs, including but not limited to, C-5 propynyl-C or C-5 propynyl-U; 2,6-diaminopurine, also known as 2-amino adenine or 2-amino-dA); hypoxanthine, pseudouridine, 2-thiopyrimidine, isocytosine (isoC), 5-methyl isoC, or isoguanine (isoG; see, e.g., U.S. Pat. No. 5,432,272). Exemplary modified pentose portions include but are not limited to, locked nucleic acid (LNA) analogs including without limitation Bz-A-LNA, 5-Me-Bz-C-LNA, dmf-G-LNA, or T-LNA (see, e.g., The Glen Report, 16(2):5, 2003; Koshkin et al., Tetrahedron 54:3607-30, 1998), or 2′- or 3′-modifications where the 2′- or 3′-position is hydrogen, hydroxy, alkoxy (e.g., methoxy, ethoxy, allyloxy, isopropoxy, butoxy, isobutoxy or phenoxy), azido, amino, alkylamino, fluoro, chloro, or bromo. Modified internucleotide linkages include phosphate analogs, analogs having achiral or uncharged intersubunit linkages (e.g., Sterchak, E. P. et al., Organic Chem., 52:4202, 1987), or uncharged morpholino-based polymers having achiral intersubunit linkages (see, e.g., U.S. Pat. No. 5,034,506). Some internucleotide linkage analogs include morpholidate, acetal, or polyamide-linked heterocycles. In one class of nucleotide analogs, known as peptide nucleic acids, including pseudo-complementary peptide nucleic acids (“PNA”), a conventional sugar and internucleotide linkage has been replaced with a 2-aminoethylglycine amide backbone polymer (see, e.g., Nielsen et al., Science, 254:1497-1500, 1991; Egholm et al., J. Am. Chem. Soc., 114: 1895-1897 1992; Demidov et al., Proc. Natl. Acad. Sci. 99:5953-58, 2002; Peptide Nucleic Acids: Protocols and Applications, Nielsen, ed., Horizon Bioscience, 2004). Certain embodiments include aspects discloses in U.S. Pat. No. 9,109,226 which is incorporated by reference in its entirety.

### Certain Inhibitor Oligonucleotides

Some embodiments of the methods and compositions provided herein include an oligonucleotide having activity to reduce or inhibit nonspecific amplification in an isothermal amplification reaction, such as a loop-mediated isothermal amplification (LAMP) reaction. The oligonucleotide can include DNA or RNA, or nucleotide analogs. In some embodiments, the oligonucleotide can have a nucleic acid sequence predicted to comprise, consist of, or consist essentially of an intra-molecular hairpin structure. As used herein, “hairpin” can refer to a secondary structure formed by a single-stranded oligonucleotide when complementary bases in a first part of the single-stranded oligonucleotide hybridize with bases in a second part of the same oligonucleotide to form a stem structure having intra-molecular base-pairing between complementary bases. In some embodiments, intra-molecular base-pairing may not occur along the oligonucleotide to form a loop structure adjacent to the stem structure. In some embodiments, the loop can include at least 1, 2, 3, 4, 5, or more consecutive nucleotides. In some embodiments, the oligonucleotide can include a portion not predicted to form part of the hairpin or loop structures. For example, some oligonucleotides can include a 5′ or 3′ terminus that extends from the hairpin structure by at least 1, 5 10, 20, 25 consecutive nucleotides, or any number in a range between any two of the foregoing number of consecutive nucleotides. In some embodiments, the predicted hairpin structure can have a predicted melting temperature (Tₘ) greater than or less than 40° C., 45° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 75° C., or a Tₘ in a range between any two of the foregoing temperatures. In some such embodiments, the predicted hairpin structure comprises or consists of a double-stranded or stem region, and a loop. In some such embodiments, the double-stranded region can include a bubble of mismatched nucleotides in which the nucleotides are non-paired. In some embodiments, a bubble can include at least or no more than 0, 1, 2, 3, 4, 5, 6, 7, 8, 9, or 10 mismatched nucleotides on one of the two strands in the double-stranded region. In some embodiments, a double stranded region can include at least or no more than 0, 1, 2, 3, or 4 bubbles. In some embodiments, an oligonucleotide having activity to reduce and/or inhibit nonspecific amplification in an isothermal amplification reaction does not specifically hybridize to a target nucleic acid in an amplification reaction, such as a LAMP reaction.

In some embodiments, an oligonucleotide having activity to reduce or inhibit nonspecific amplification in an isothermal amplification reaction, such as an inhibitor oligonucleotide, can comprise, consist of, or consist essentially of a nucleic acid sequence having a sequence identity with a certain nucleic acid sequence. In some embodiments, an inhibitor oligonucleotide can comprise, consist of, or consist essentially of a nucleic acid sequence having a sequence identity with a nucleic acid sequence selected from any one of SEQ ID NOs:01-15 of at least 80%, 85%, 90%, 95%, 96%, 97%, 98%, 99%, 100%, or any percentage within a range of any two of the foregoing percentages. In some embodiments, an inhibitor oligonucleotide can comprise, consist of, or consist essentially of a nucleic acid sequence having a sequence identity with a nucleic acid sequence selected from any one of SEQ ID NOs:01-10 of at least 80%, 85%, 90%, 95%, 96%, 97%, 98%, 99%, 100%, or any percentage within a range of any two of the foregoing percentages. In some embodiments, an inhibitor oligonucleotide can comprise, consist of, or consist essentially of a nucleic acid sequence having a sequence identity with the nucleotide sequence of SEQ ID NO:09 of at least 80%, 85%, 90%, 95%, 96%, 97%, 98%, 99%, 100%, or any percentage within a range of any two of the foregoing percentages. In some embodiments, an inhibitor oligonucleotide can comprise, consist of, or consist essentially of a nucleic acid sequence having a sequence identity with the nucleotide sequence of SEQ ID NO:01 of at least 80%, 85%, 90%, 95%, 96%, 97%, 98%, 99%, 100%, or any percentage within a range of any two of the foregoing percentages. In some embodiments, an inhibitor oligonucleotide can comprise, consist of, or consist essentially of a nucleic acid sequence having a sequence identity with the nucleotide sequence of SEQ ID NO:02 of at least 80%, 85%, 90%, 95%, 96%, 97%, 98%, 99%, 100%, or any percentage within a range of any two of the foregoing percentages.

In some embodiments, an oligonucleotide having activity to reduce or inhibit nonspecific amplification in an isothermal amplification reaction, such as an inhibitor oligonucleotide, can comprise, consist of, or consist essentially of a nucleic acid sequence capable of hybridizing to the complement of a certain nucleic acid sequence. For example, a nucleic acid can have a sequence capable of hybridizing to another nucleic acid under predetermined conditions. Hybridization includes a process during which, under suitable conditions, two polynucleotides having sufficiently complementary sequences are capable of forming a double strand with stable and specific hydrogen bonds. A probe polynucleotide “hybridizable” to target polynucleotide is capable of hybridizing with the target polynucleotide under hybridization conditions that can be determined in each case in a known manner. Hybridization is more specific when it is carried out with higher stringency. The stringency is defined in particular depending on the base composition of a probe/target duplex, as well as by the degree of mismatch between two nucleic acids. Stringency can also be a function of reaction parameters, such as concentration and type of ionic species present in the hybridization solution, the nature and concentration of denaturing agents or hybridization temperature. The stringency of the conditions under which a hybridization reaction must be carried out depend principally the probe/targets used. In general, depending on the length of the nucleic acids used, the temperature for the hybridization reaction is between approximately 20° C. and 65° C., in particular between 35° C. and 65° C. in saline at a concentration of about 0.08 to 1 M.

In some embodiments, an inhibitor oligonucleotide can comprise, consist of, or consist essentially of a nucleic acid sequence capable of hybridizing to the complement of a nucleic acid having a sequence selected from any one of SEQ ID NOs:01-15. In some embodiments, an inhibitor oligonucleotide can comprise, consist of, or consist essentially of a nucleic acid sequence capable of hybridizing to the complement of a nucleic acid having a sequence selected from any one of SEQ ID NOs:01-10. In some embodiments, an inhibitor oligonucleotide can comprise, consist of, or consist essentially of a nucleic acid sequence capable of hybridizing to the complement of a nucleic acid having a sequence of SEQ ID NO:09. In some embodiments, an inhibitor oligonucleotide can comprise, consist of, or consist essentially of a nucleic acid sequence capable of hybridizing to the complement of a nucleic acid having a sequence of SEQ ID NO:01. In some embodiments, an inhibitor oligonucleotide can comprise, consist of, or consist essentially of a nucleic acid sequence capable of hybridizing to the complement of a nucleic acid having a sequence of SEQ ID NO:02.

In some embodiments, an inhibitor oligonucleotide can comprise, consist of, or consist essentially of a nucleic acid sequence selected from any one of SEQ ID NOs:01-15. In some embodiments, an inhibitor oligonucleotide can comprise, consist of, or consist essentially of a nucleic acid sequence selected from any one of SEQ ID NOs:01-10. In some embodiments, an inhibitor oligonucleotide can comprise, consist of, or consist essentially of the nucleotide sequence of SEQ ID NO:09. In some embodiments, an inhibitor oligonucleotide can comprise, consist of, or consist essentially of the nucleotide sequence of SEQ ID NO:01. In some embodiments, an inhibitor oligonucleotide can comprise, consist of, or consist essentially of the nucleotide sequence of SEQ ID NO:02.

In some embodiments, an oligonucleotide lacks a nucleotide comprising uracil or inosine.

In some embodiments, an inhibitor oligonucleotide can comprise a blocking moiety. For example, an inhibitor oligonucleotide can include a blocking moiety that prevents extension of the oligonucleotide. As used herein, “blocking moiety” when used in reference to a nucleotide analog, refers to a part of the nucleotide analog that inhibits or prevents the nucleotide analog from forming a covalent linkage to a second nucleotide analog. For example, in the case of nucleotide analogs having a pentose moiety, a blocking moiety can prevent formation of a phosphodiester bond between the 3′ oxygen of the nucleotide analog and the 5′ phosphate of the second nucleotide analog. The blocking moiety can be part of a nucleotide analog that is a monomer unit present in a nucleic acid polymer or the blocking moiety can be a part of a free nucleotide analog (e.g. a nucleotide triphosphate). The blocking moiety that is part of a nucleotide analog can be reversible, such that the blocking moiety can be removed or modified to render the nucleotide analog capable of forming a covalent linkage to a second nucleotide analog. Particularly useful reversible blocking moieties are phosphates, phosphoesters, alkyl azides, acetals, esters, or ethers or the like. In some embodiments, a blocking moiety, such as a reversible blocking moiety, can be attached to the 3′ position or 2′ position of a pentose moiety of a nucleotide analog. In some embodiments, the blocking moiety can be readily removed from the inhibitor oligonucleotide. In some embodiments, the inhibitor oligonucleotide can be phosphorylated, for example at the 3′ end of the oligonucleotide. Examples of blocking moieties are disclosed in U.S. 20180312917, which is incorporated herein by reference in its entirety.

### Certain Compositions

Some embodiments of the methods and compositions provided herein include aqueous solutions. In some embodiments an aqueous solution can include a first inhibitor oligonucleotide, such as an inhibitor oligonucleotide comprising a hairpin, as provided herein. In some embodiments, the first inhibitor oligonucleotide does not specifically hybridize to the target nucleic acid. In some embodiments, the first inhibitor oligonucleotide has activity to reduce the level of a nonspecific amplification product of the LAMP reaction compared to the level of a nonspecific amplification product of a LAMP reaction performed in the absence of the first inhibitor oligonucleotide. In some embodiments, an aqueous solution further comprises a set of LAMP primers sufficient to perform a LAMP reaction of a target nucleic acid. In some embodiments, an aqueous solution further comprises a polymerase, such as a polymerase suitable for a LAMP reaction.

In some embodiments, the 3′ end of the first inhibitor oligonucleotide comprises a blocking moiety which inhibits polymerase extension of the first inhibitor oligonucleotide. Examples of blocking moieties are provided herein, and include a phosphate, a C3 spacer, an amine, biotin, or an inverted base. In some embodiments, the 3′ end of the first inhibitor oligonucleotide is phosphorylated.

In some embodiments, the first inhibitor oligonucleotide lacks a nucleotide comprising uracil or inosine.

In some embodiments, the hairpin structure can have a predicted melting temperature (Tₘ) greater than or less than 40° C., 45° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., or 75° C., or a Tₘ in a range between any two of the foregoing temperatures. In some embodiments, the hairpin has a Tₘ less than about 65° C. In some embodiments, the hairpin has a Tₘ less than about 55° C. In some embodiments, the hairpin has a Tₘ in the range of about 50° C. to about 60° C. In some embodiments, the hairpin has a Twin the range of 50° C. to 60° C.

In some embodiments, a 3′ terminal nucleotide of the first inhibitor oligonucleotide is single-stranded, and a nucleotide of the first inhibitor oligonucleotide consecutive with the 3′ terminal nucleotide is double-stranded.

In some embodiments, the hairpin of the first inhibitor oligonucleotide comprises a loop comprising or consisting of 2, 3, 4, 5, 6, 7, 8, 9, 10, 11, 12, 13, 14, 15, 16, 17, 18, 19, 20, 25, 30, 35, 40, 45, or 50 consecutive single-stranded nucleotides. In some embodiments, the hairpin comprises a loop comprising or consisting of 3 consecutive single-stranded nucleotides.

In some embodiments, the first inhibitor oligonucleotide comprises, consists of, or consists essentially of: a nucleic acid having at least 90% sequence identity with the nucleotide sequence selected from any one of SEQ ID NOs:01-15; a nucleic acid capable of hybridizing or configured to hybridize to the complement of a nucleic acid having the nucleotide sequence selected from any one of SEQ ID NOs:01-15; or a nucleic acid having the nucleotide sequence selected from any one of SEQ ID NOs:01-15. In some embodiments, the first inhibitor oligonucleotide comprises, consists of, or consists essentially of: a nucleic acid having at least 90% sequence identity with the nucleotide sequence of SEQ ID NO:09; a nucleic acid capable of hybridizing or configured to hybridize to the complement of a nucleic acid having the nucleotide sequence of SEQ ID NO:09; or a nucleic acid having the nucleotide sequence of SEQ ID NO:09. In some embodiments, the first inhibitor oligonucleotide comprises, consists of, or consists essentially of: a nucleic acid having at least 90% sequence identity with the nucleotide sequence of SEQ ID NO:01; a nucleic acid capable of hybridizing or configured to hybridize to the complement of a nucleic acid having the nucleotide sequence of SEQ ID NO:01; or a nucleic acid having the nucleotide sequence of SEQ ID NO:01. In some embodiments, the first inhibitor oligonucleotide comprises, consists of, or consists essentially of: a nucleic acid having at least 90% sequence identity with the nucleotide sequence of SEQ ID NO:02; a nucleic acid capable of hybridizing or configured to hybridize to the complement of a nucleic acid having the nucleotide sequence of SEQ ID NO:02; or a nucleic acid having the nucleotide sequence of SEQ ID NO:02.

In some embodiments, the LAMP reagent mix further comprises a second inhibitor oligonucleotide.

In some embodiments, the 3′ end of the second inhibitor oligonucleotide comprises a blocking moiety which inhibits polymerase extension of the second inhibitor oligonucleotide.

In some embodiments, the 3′ end of the second inhibitor oligonucleotide is phosphorylated.

In some embodiments, a 3′ terminal nucleotide of the second inhibitor oligonucleotide is single-stranded, and a nucleotide of the second inhibitor oligonucleotide consecutive with the 3′ terminal nucleotide is double-stranded.

In some embodiments, a ratio between the first inhibit oligonucleotide and the second inhibitor oligonucleotide in the aqueous solution is in a range between 1:10 and 1:1. In some embodiments, the ratio is 1:5 or 1:5 or is about 1:5 or 1:5.

In some embodiments, the second inhibitor oligonucleotide comprises, consists of, or consists essentially of: a nucleic acid having at least 90% sequence identity with the nucleotide sequence selected from any one of SEQ ID NOs:01-15; a nucleic acid capable of hybridizing or configured to hybridize to the complement of a nucleic acid having a nucleotide sequence selected from any one of SEQ ID NOs:01-15; or a nucleic acid having the nucleotide sequence selected from any one of SEQ ID NOs:01-15. In some embodiments, the second inhibitor oligonucleotide comprises, consists of, or consists essentially of: a nucleic acid having at least 90% sequence identity with the nucleotide sequence of SEQ ID NO:09; a nucleic acid capable of hybridizing or configured to hybridize to the complement of a nucleic acid having a nucleotide sequence of SEQ ID NO:09; or a nucleic acid having the nucleotide sequence of SEQ ID NO:09. In some embodiments, the second inhibitor oligonucleotide comprises, consists of, or consists essentially of: a nucleic acid having at least 90% sequence identity with the nucleotide sequence of SEQ ID NO:01; a nucleic acid capable of hybridizing or configured to hybridize to the complement of a nucleic acid having the nucleotide sequence of SEQ ID NO:01; or a nucleic acid having the nucleotide sequence of SEQ ID NO:01. In some embodiments, the second inhibitor oligonucleotide comprises, consists of, or consists essentially of: a nucleic acid having at least 90% sequence identity with the nucleotide sequence of SEQ ID NO:02; a nucleic acid capable of hybridizing or configured to hybridize to the complement of a nucleic acid having the nucleotide sequence of SEQ ID NO:02; or a nucleic acid having the nucleotide sequence of SEQ ID NO:02.

Some embodiments also include an aqueous solution comprising a crowding agent. In some embodiments, the crowding agent is selected from polyethylene glycol (PEG), dextran, polyvinyl alcohol, polyvinyl pyrrolidone, or Ficoll. In some embodiments, the crowding agent is selected from PEG-35K, PEG-8K or Ficoll-400K. In some embodiments, the crowding agent comprises PEG-35K.

In some embodiments, the polymerase is suitable for a LAMP reaction. In some embodiments, the polymerase comprises a strand displacing activity. In some embodiments, the polymerase is selected from Bst large fragment, Bca (exo-), Vent, Vent (exo), Deep Vent, Deep Vent (exo-), phi29 phage, MS-2 phage, Taq, Z-Taq, KOD, Klenow fragment, Bst 2.0, Bst 3.0, a Bst derivative, a Bsu polymerase, a Gsp polymerase, or a Sau polymerase or any combination thereof. In some embodiments, the polymerase comprises a Bst large fragment.

In some embodiments, the first inhibitor oligonucleotide has a concentration in a range from 0.1 μM to 20 μM or about 0.1 μM to about 20 μM. In some embodiments, the second inhibitor oligonucleotide has a concentration in a range from 0.1 μM to 20 μM or about 0.1 μM to about 20 μM.

Some embodiments also include an aqueous solution comprising a plurality of different sets of LAMP primers, each set sufficient to perform a LAMP reaction of a different target nucleic acid. In some embodiments, a primer of the set of LAMP primers comprises the nucleotide sequence selected from any one of SEQ ID NOs:19-162. In some embodiments, the set of LAMP primers comprises a F3 primer, a B3 primer, a FIP primer, a BIP primer, a LF primer and a LB primer, each primer having the nucleotide sequence selected from any one of SEQ ID NOs:19-162.

In some embodiments, the target nucleic acid is a nucleic acid from a virus or organism selected from Dengue virus, Influenza A virus strain H3N1, Influenza A virus strain H3N2, Haemophilus influenzae, Hepatitis A virus, Hepatitis B virus, Hepatitis C virus, Human immunodeficiency virus-1, Plasmodium spp, Bacteriophage MS2, Parvovirus B19, Respiratory syncytial virus, Salmonella typhimurium, strain LT2, Mycobacterium tuberculosis, or Zika virus.

In some embodiments, the first inhibitor oligonucleotide has activity to increase a critical time (Ct) value for the amplification of a false positive in the LAMP reaction compared to a Ct value for the amplification of the false positive in a LAMP reaction performed in the absence of the first inhibitor oligonucleotide. In some embodiments, the increase is at least 2-, 3-, 4-, 5-, 6-, 7-, 8-, 9- or 10— fold. In some embodiments, the increase is at least 2-fold. In some embodiments, the increase is at least 3-fold. In some embodiments, the increase is at least 1, 2, 3, 4, 5, 6, 7, 8, 9, 10, 11, 12, 13, 14, 15, 16, 17, 18, 19, or 20 minutes.

### Certain Methods for Reducing Nonspecific Amplification

Some embodiments of the methods and compositions provided herein include a method of reducing nonspecific amplification in an isothermal amplification reaction, such as a LAMP reaction. In some embodiments, the LAMP reaction specifically amplifies a target nucleic acid. In some embodiments, the level of nonspecific amplification in a LAMP reaction performed in the presence of an inhibitor oligonucleotide provided herein is reduced compared to the level of nonspecific amplification in a LAMP reaction performed in the absence of the inhibitor oligonucleotide. In some embodiments, nonspecific amplification can be detected as false positives in an amplification reaction.

Some embodiments of a method of reducing nonspecific amplification in a LAMP reaction can include providing a LAMP reagent mix. In some embodiments, a LAMP reagent mix can include reagents sufficient to amplify a target nucleic acid. Examples of such reagents include a set of LAMP primers sufficient to perform a LAMP reaction of a target nucleic acid, such as a F3 primer, a B3 primer, a FIP primer, a BIP primer, a LF primer, and a LB primer; and a polymerase, such as a polymerase comprising a strand displacing activity. In some embodiments, the polymerase is selected from Bst large fragment, Bca (exo-), Vent, Vent (exo-), Deep Vent, Deep Vent (exo-), phi29 phage, MS-2 phage, Taq, Z-Taq, KOD, or Klenow fragment, or any combination thereof. In some embodiments, a polymerase can be selected from Bst 2.0 (NEB), Bst 3.0 (NEB), a Bst derivative, a Bsu polymerase, a Gsp polymerase, or a Sau polymerase. In some embodiments, the polymerase comprises a Bst large fragment.

In some embodiments, the reagent mix can include a crowding agent. Examples of crowding agents include polyethylene glycol (PEG) such as PEG1450, PEG3000, PEG8000 (PEG-8K), PEG10000, PEG14000, PEG15000, PEG20000, PEG250000, PEG30000, PEG35000 (PEG-35K), PEG40000 (PEG-400k); dextran; polyvinyl alcohol; polyvinyl pyrrolidone; or Ficoll. In some embodiments, the crowding agent is selected from PEG-35K, PEG-8K or Ficoll 400K. In some embodiments, the crowding agent comprises PEG-35K. In some embodiments, the crowding agent is present in the LAMP reaction at a concentration between 1 to 12% by weight or by volume of the reaction, such as between any two concentration values selected from 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10.0%, 10.5%, 11.0%, 11.5%, 12.0%, 12.5%, 13%, 13.5%, 14%, 14.5%, 15%, or 20%.

In some embodiments, the LAMP reaction in performed in a presence of an inhibitor oligonucleotide. In some embodiments, the inhibitor oligonucleotide can comprise, consist of, or consist essentially of a nucleic acid sequence having a sequence identity with a nucleic acid sequence selected from SEQ ID NOs:01-15 of at least 80%, 85%, 90%, 95%, 96%, 97%, 98%, 99%, 100%, or any percentage within a range of any two of the foregoing percentages. In some embodiments, the inhibitor oligonucleotide can comprise, consist of, or consist essentially of a nucleic acid sequence having a sequence identity with a nucleic acid sequence selected from SEQ ID NOs:01-10 of at least 80%, 85%, 90%, 95%, 96%, 97%, 98%, 99%, 100%, or any percentage within a range of any two of the foregoing percentages. In some embodiments, the inhibitor oligonucleotide can comprise, consist of, or consist essentially of a nucleic acid sequence having a sequence identity with the nucleotide sequence of SEQ ID NO:09 of at least 80%, 85%, 90%, 95%, 96%, 97%, 98%, 99%, 100%, or any percentage within a range of any two of the foregoing percentages. In some embodiments, the inhibitor oligonucleotide can comprise, consist of, or consist essentially of a nucleic acid sequence having a sequence identity with the nucleotide sequence of SEQ ID NO:01 of at least 80%, 85%, 90%, 95%, 96%, 97%, 98%, 99%, 100%, or any percentage within a range of any two of the foregoing percentages. In some embodiments, the inhibitor oligonucleotide can comprise, consist of, or consist essentially of a nucleic acid sequence capable of hybridizing to the complement of a nucleic acid having a sequence selected from any one of SEQ ID NOs:01-15. In some embodiments, the inhibitor oligonucleotide can comprise, consist of, or consist essentially of a nucleic acid sequence capable of hybridizing or configured to hybridize to the complement of a nucleic acid having a sequence selected from any one of SEQ ID NOs:01-10. In some embodiments, the inhibitor oligonucleotide can comprise, consist of, or consist essentially of a nucleic acid sequence capable of hybridizing or configured to hybridize to the complement of a nucleic acid having a sequence of SEQ ID NO:09. In some embodiments, the inhibitor oligonucleotide can comprise, consist of, or consist essentially of a nucleic acid sequence capable of hybridizing or configured to hybridize to the complement of a nucleic acid having a sequence of SEQ ID NO:01. In some embodiments, the inhibitor oligonucleotide can comprise, consist of, or consist essentially of a nucleic acid sequence selected from any one of SEQ ID NOs:01-15. In some embodiments, the inhibitor oligonucleotide can comprise, consist of, or consist essentially of a nucleic acid sequence selected from any one of SEQ ID NOs:01-10. In some embodiments, the inhibitor oligonucleotide can comprise, consist of, or consist essentially of the nucleotide sequence of SEQ ID NO:09. In some embodiments, the inhibitor oligonucleotide can comprise, consist of, or consist essentially of the nucleotide sequence of SEQ ID NO:01. In some embodiments, the inhibitor oligonucleotide can include a blocking moiety to prevent extension. In some embodiments, the inhibitor oligonucleotide can be phosphorylated, for example at the 3′ end of the oligonucleotide.

In some embodiments, the concentration of an inhibitor oligonucleotide in a LAMP reaction can be in a range from about 0.01 μM to 100 μM, or from about 0.1 μM to about 20 μM or from 0.01 μM to 100 μM, or from 0.1 μM to about 20 μM. In some embodiments, the concentration of an inhibitor oligonucleotide in a LAMP reaction can be 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, or 20 μM or within a range defined by any two of the aforementioned concentrations.

In some embodiments, a LAMP reaction can be performed in the presence of a combination of at least two inhibitor oligonucleotides. In some embodiments, the at least two inhibitor oligonucleotides each comprise, consist of, or consist essentially of a nucleic acid having at least 80%, 85%, 90%, 95%, 96%, 97%, 98%, 99%, 100%, or any percentage within a range of any two of the foregoing percentages, sequence identity with a nucleic acid sequence selected from any one of SEQ ID NOs:01-15. In some embodiments, the at least two inhibitor oligonucleotides each comprise, consist of, or consist essentially of a nucleic acid capable of hybridizing or configured to hybridize to the complement of a nucleic acid having a sequence selected from any one of SEQ ID NOs:01-15. In some embodiments, the at least two inhibitor oligonucleotides comprise an inhibitor oligonucleotide having the nucleotide sequence of SEQ ID NO:01, and an inhibitor oligonucleotide having the nucleotide sequence of SEQ ID:02. In some embodiments, one or more of the at least two inhibitor oligonucleotides is phosphorylated.

In some embodiments, a ratio of concentrations of the at least two inhibitor oligonucleotides in a LAMP reaction, such as in a LAMP reagent mix, can be in a range between 1:10 and 1:1, 1:8 and 1:2, or 1:6 and 1:4. In some embodiments, the ratio of concentrations of the at least two inhibitor oligonucleotides in a LAMP reaction, such as in a LAMP reagent mix, can be 1:5.

In some embodiments, the LAMP reagent mix can include a single set of LAMP primers sufficient to amplify a single target nucleic acid. In some embodiments, the LAMP reagent mix can include a plurality of sets of LAMP primers, each set sufficient to amplify a single different target nucleic acid. In some embodiments, a primer of the set of LAMP primers can comprise, consist of, or consist essentially of a nucleic acid sequence having at least 80%, 85%, 90%, 95%, 96%, 97%, 98%, 99%, 100%, or any percentage within a range defined by any two of the foregoing percentages, sequence identity with a nucleic acid sequence selected from any one of SEQ ID NOs:19-156.

In some embodiments, a target nucleic acid can include any nucleic acid sequence of interest to be amplified in a LAMP reaction. Examples of target nucleic acids include nucleic acid sequences from a virus or organism such as Dengue virus, Influenza A virus strain H3N1, Influenza A virus strain H3N2, Haemophilus influenzae, Hepatitis A virus, Hepatitis B virus, Hepatitis C virus, Human immunodeficiency virus-1, Plasmodium spp, Bacteriophage MS2, Parvovirus B19, Respiratory syncytial virus, Salmonella typhimurium, strain LT2, Mycobacterium tuberculosis, or Zika virus.

In some embodiments, a LAMP reaction, such as a LAMP reagent mix, may contain any desired concentration of each component and/or reagent sufficient to achieve the desired results. Such individual components may be individually or separately optimized for this purpose. For multiplex reactions, total primer concentrations may also be optimized as necessary for the individual assay. For multiplex assays or reactions, concentrations of reagents maybe kept as for single assays, or may be altered to suit the particular application. In some embodiments, concentrations of reagents may be used as described herein for a standard LAMP reaction, with each set of primers or probes representing 1/n of the total, where n is the number of targets and respective primer sets being evaluated in a particular analysis.

In some embodiments, a LAMP reaction may be performed in any reaction volume, for example a reaction volume can be at least 0.25 μL, 0.5 μL, 1 μL, 2 μL, 3 μL, 4 μL, 5 μL, 10 μL, 15 μL, 20 μL, 25 μL, 30 μL, 35 μL, 40 μL, 45 μL, 50 μL, 60 μL, 70 μL, 80 μL, 90 μL, 100 μL, 125 μL, 150 μL, 175 μL, 200 μL, 250 μL, 300 μL, 350 μL, 400 μL, 450 μL, 500 μL, or 1 mL, or any volume in a range defined by any two of the foregoing volumes.

In some embodiments, a plurality of LAMP reactions can be performed in the presence and absence of a target nucleic acid. For example, in a plurality of LAMP reactions a negative control may contain no target nucleic acid. In some such embodiments, the activity of an inhibitor oligonucleotide can be readily observed, for example, the amplification of a false positive in a LAMP reaction in the absence of the target nucleic acid and presence of the inhibitor oligonucleotide is decreased compared to amplification of a false positive in a LAMP reaction in the absence of the target nucleic acid and the inhibitor oligonucleotide. In some embodiments, the reduction comprises an increase in a critical time (Ct) value for the amplification of a false positive in a LAMP reaction in the absence of the target nucleic acid and presence of the inhibitor oligonucleotide compared to a Ct value for the amplification of a false positive in a LAMP reaction in the absence of the target nucleic acid and the inhibitor oligonucleotide. In some embodiments, the increase in a Ct value is at least 2-fold. In some embodiments, the increase in a Ct value is at least 3-fold. In some embodiments, the increase is at least 1, 2, 3, 4, 5, 6, 7, 8, 9, 10, 11, 12, 13, 14, 15, 16, 17, 18, 19, or 20 minutes.

In some embodiments, an amplification product of the LAMP reaction is detected by changes in a signal selected from an optical signal, a pH signal, and an electrical signal. In some embodiments, an amplification product of the LAMP reaction is detected by changes in an electrical signal. Example systems, methods and devices that can be used to readily detect LAMP amplification products, such as by electrical signals, are disclosed in U.S. Pat. No. 9,506,908; U.S. Pat. Pub. No. 2017/0114398; U.S. Pat. Pub. No. 2016/0097742; Int. Pat. Pub. No. WO/2016/057422; and Int. Pat. Pub. No. WO/2018/057647 which are each expressly incorporated by reference in its entirety.

In some embodiments, analysis of data may be performed using any applicable statistical methods, such as Ct value, in order to reflect the time taken to reach a positive signal threshold. These values may be used to plot calibration curves as a function of target copy number input load for each separate target in the reference sample. Means and variances of the rates of concurrence may be evaluated for significance with the Student's t-test with determination of effect size along with p-values and standard deviations within each experiment for duplicates and triplicates (intra-assay) and between independent experiments (inter-assay).

### Certain Systems

Some embodiments of the methods and compositions provided herein include a system for detecting a target nucleic acid in a LAMP reaction. Some such systems can include a vessel comprising an aqueous solution provided herein comprising a LAMP reagent mix. The vessel can include a container configured to contain the LAMP reagent mix. Examples of vessels include wells, channels, passageways, conduits, plates, or tubes. The vessel can be in contact with a heating source, configured to heat the LAMP reagents to a temperature sufficient to perform the LAMP reaction. The LAMP reaction mix can contain a set of LAMP primers sufficient to perform a LAMP reaction of a target nucleic acid as provided herein; a polymerase as provided herein, and the inhibitor oligonucleotide as provided herein. In some embodiments, the reagent mix can include a crowding agent. Examples of crowding agents include polyethylene glycol (PEG), dextran, polyvinyl alcohol, polyvinyl pyrrolidone, or Ficoll.

In some embodiments, the system can include a detector configured to detect an amplification product in the vessel. In some embodiments, the detector is configured to detect a change in an electrical signal, pH, or an optical signal. In some embodiments, the detector is configured to detect a change in an electrical signal. Example systems, methods and devices that can be used to readily detect LAMP amplification products, such as by electrical signals, are disclosed in U.S. Pat. No. 9,506,908; U.S. Pat. Pub. No. 2017/0114398; U.S. Pat. Pub. No. 2016/0097742; Int. Pat. Pub. No. WO/2016/057422; and Int. Pat. Pub. No. WO/2018/057647 which are each expressly incorporated by reference in its entirety.

### Certain Kits

Some embodiments of the methods and compositions provided herein include a kit. In some embodiments, a kit can include an inhibitor oligonucleotide provided herein, and at least one reagent for performing a LAMP reaction, such as a polymerase comprising a strand displacement activity, and a set of loop-mediated isothermal amplification (LAMP) primers sufficient to perform a LAMP reaction of a target nucleic acid. In some embodiments, a kit can include an aqueous solution provided herein.

In some embodiments, the kit can also include at least a second inhibitor oligonucleotide. In some embodiments, the at least a second inhibitor oligonucleotide can comprise, consist of, or consist essentially of a nucleic acid having at least 80%, 85%, 90%, 95%, 96%, 97%, 98%, 99%, or 100%, or any percentage within a range defined by any two of the foregoing percentages, sequence identity with a nucleic acid sequence selected from any one of SEQ ID NOs:01-15. In some embodiments, the at least a second inhibitor oligonucleotide can comprise, consist of, or consist essentially of a nucleic acid capable of hybridizing to the complement of a nucleic acid having a sequence selected from any one of SEQ ID NOs:01-15.

In some embodiments, an inhibitor oligonucleotide having the nucleotide sequence of SEQ ID NO:01, and an inhibitor oligonucleotide having the nucleotide sequence of SEQ ID:02. In some embodiments, the ratio between the oligonucleotide having the nucleic acid sequence of SEQ ID NO:02 and the inhibitory oligonucleotide having the nucleic acid sequence of SEQ ID NO:01 in the LAMP reagent mix is in a range between 1:10 and 1:1. In some embodiments, the ratio is 1:5.

In some embodiments, the kit can also include a plurality of different sets of LAMP primers, each set sufficient to perform a LAMP reaction of a different target nucleic acid. In some embodiments, the set of LAMP primers comprises at least 4 different primers. In some embodiments, the set of LAMP primers comprises at least 6 different primers. In some embodiments, a primer of the set of LAMP primers comprises a nucleic acid sequence selected from any one of SEQ ID NOs:19-156.

In some embodiments, the polymerase is selected from Bst large fragment, Bca (exo-), Vent, Vent (exo-), Deep Vent, Deep Vent (exo-), phi29 phage, MS-2 phage, Taq, Z-Taq, KOD, Klenow fragment, Bst 2.0 (NEB), Bst 3.0 (NEB), a Gsp polymerase, a Bst derivative, a Bsu polymerase, or a Sau polymerase, or any combination thereof.

In some embodiments, the reagent mix comprises a crowding agent. In some embodiments, the crowding agent can include one or more of polyethylene glycol (PEG), dextran, polyvinyl alcohol, polyvinyl pyrrolidone, or Ficoll.

### EXAMPLES

### Example 1—Activity of Phosphorylated and Extended HAVFIP1 Variants

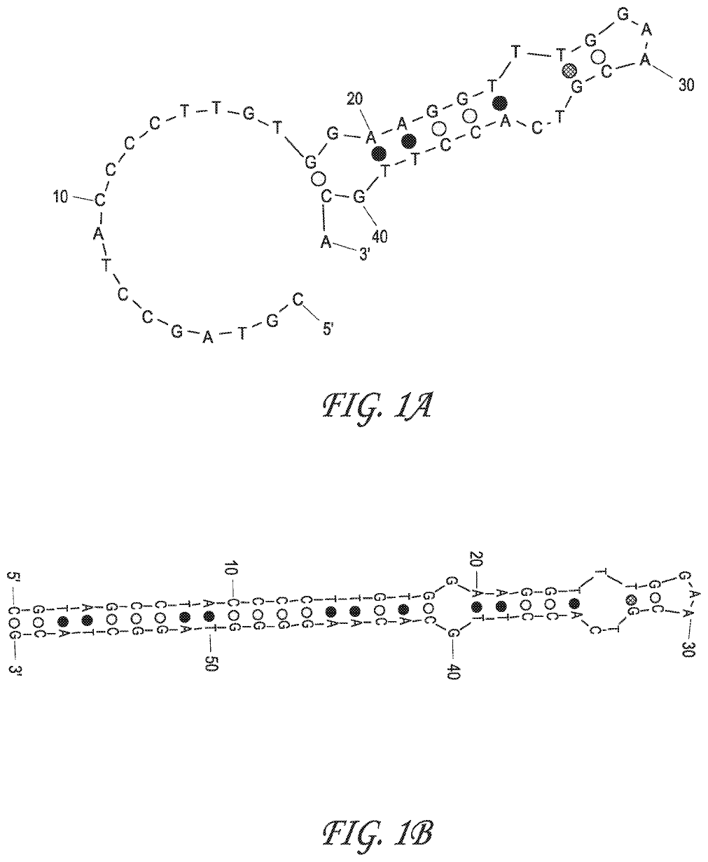

An oligonucleotide, HAVFIP1, was discovered to inhibit nonspecific amplification in LAMP reactions. Activity of phosphorylated or extended variants of HAVFIP1 to inhibit nonspecific amplification in LAMP with an HCV LAMP primer set in the presence of an HCV target, was tested. Thermodynamic modeling of the HAVFIP1 oligonucleotide predicted that the oligonucleotide formed a hairpin with a long pseudo-double stranded region near its 3′ end (FIG. 1A). The pseudo-double stranded region may be extended by 16 bases to produce the structure shown in FIG. 1B. Tested variants included extension of the pseudo-double stranded region. HAVFIP1 variants included a HAVFIP1 oligonucleotide having a phosphorylated 3′ end, and HAVFIP1 oligonucleotides having a phosphorylated 4-, 8-, 12- or 16-nucleotide 3′ extension. The variants are listed in TABLE 1.

TABLE 1

| Oligonucleotide | SEQ ID NO: |
|---|---|
| HAVF IP 1 | SEQ ID NO: 01 |
| HAVFIP1 Phos3′ | SEQ ID NO: 01 |
| HAVFIP1_4b | SEQ ID NO: 02 |
| HAVFIP1_8b | SEQ ID NO: 06 |
| HAVFIP1_12b | SEQ ID NO: 07 |
| HAVFIP1_16b | SEQ ID NO: 08 |

Reactions were prepared in WarmStart LAMP Master Mix (New England Biolabs, Ipswich, Mass.). This contained 20 mM Tris-HCl (pH 8.8 at 25° C.), 8 mM magnesium sulfate, 50 mM potassium chloride, 10 mM ammonium sulfate, 0.1% Tween-20, 8 U WarmStart Bst 2.0 DNA Polymerase, 7.5 U WarmStart RTx Reverse Transcriptase, and 5.6 mM total dNTPs (1.4 mM each). Additional components included 0.5 U Antarctic Thermolabile UDG (NEB), 0.7 mM dUTP (MilliporeSigma), 1× EvaGreen intercalating dye (Biotium, Fremont, Calif.), and primer set “HCV Pr5” (comprised of 40 μmol each FIP/BIP, 10 μmol each LF/LB, and 5 μmol each F3/B3). Positive samples contained an equimolar mixture of synthetic DNA sequences derived from HCV genotypes 1, 2, and 3 (“HCV Synth”; 3.33×10⁵ copies/reaction each sequence). Negative samples ‘no template controls’ (NTCs) contained no templates. Positive and negative samples were tested with 0, 2, 5, or 10 μM of each HAVFIP1 variant from TABLE 1. The total reaction volume in each case was 25 μL. The LAMP reactions were run at 65° C. for 120 minutes on an Applied Biosystems QuantStudio 3 (QS3) thermocycler. Fluorescence was measured once per minute to assess reaction progress. Target nucleic acids including LAMP primer sets are listed in TABLES 14-19. Results are summarized in TABLE 2. “Neat” conditions contain no HAVFIP1 variant, while “FIP” in each table refers to the HAVFIP1 variant being tested. “# of Amps” refers to the number of replicates that amplified from that test condition. Three replicates were run for each condition with template, and NTCs were tested with 6 replicates each.

TABLE 2

| HAVFIP1 | | Ct Mean | Ct | Ct | # of |
|---|---|---|---|---|---|
| variant | Sample Name | (min) | SD | % RSD | Amps |
| HAV FIP1 | Avg. HCV | 16.51 | 0.18 | 1.08 | |
| | Synth Neat | | | | |
| | HCV Synth + | 20.34 | 0.04 | 0.22 | 3 |
| | 2 μm FIP | | | | |
| | HCV Synth + | 33.37 | 0.14 | 0.42 | 3 |
| | 5 μm FIP | | | | |
| | HCV Synth + | 61.45 | 1.39 | 2.26 | 3 |
| | 10 μm FIP | | | | |
| | Avg. NTCs Neat | 42.69 | 1.32 | 3.08 | |
| | NTCs + | 96.85 | 12.07 | 12.47 | 4 |
| | 2 μm FIP | | | | |
| | NTCs + | | | | 0 |
| | 5 μm FIP | | | | |
| | NTCs + | | | | 0 |
| | 10 μm FIP | | | | |
| HAV FIP1_4b | Avg. HCV | 16.51 | 0.18 | 1.08 | |
| ext_Phos3′ | Synth Neat | | | | |
| | HCV Synth + | 17.07 | 0.22 | 1.31 | 3 |
| | 2 μm FIP | | | | |
| | HCV Synth + | 17.70 | 0.15 | 0.86 | 3 |
| | 5 μm FIP | | | | |
| | HCV Synth + | 20.45 | 0.19 | 0.95 | 3 |
| | 10 μm FIP | | | | |
| | Avg. NTCs Neat | 42.69 | 1.32 | 3.08 | |
| | NTCs + | 71.49 | 18.76 | 26.24 | 5 |
| | 2 μm FIP | | | | |
| | NTCs + | 118.51 | | | 1 |
| | 5 μm FIP | | | | |
| | NTCs + | 76.59 | 16.33 | 21.32 | 2 |
| | 10 μm FIP | | | | |
| HAV FIP1_12b | Avg. HCV | 16.51 | 0.18 | 1.08 | |
| ext_Phos3′ | Synth Neat | | | | |
| | HCV Synth + | 25.51 | 0.29 | 1.15 | 3 |
| | 2 μm FIP | | | | |
| | HCV Synth + | 49.81 | 1.09 | 2.19 | 3 |
| | 5 μmFIP | | | | |
| | HCV Synth + | 106.29 | 1.22 | 1.14 | 3 |
| | 10 μm FIP | | | | |
| | Avg. NTCs Neat | 42.69 | 1.32 | 3.08 | |
| | NTCs + | | | | 0 |
| | 2 μm FIP | | | | |
| | NTCs + | | | | 0 |
| | 5 μm FIP | | | | |
| | NTCs + | | | | 0 |
| | 10 μm FIP | | | | |
| HAV FIP1_ | Avg. HCV | 16.51 | 0.18 | 1.08 | |
| Phos3′ | Synth Neat | | | | |
| | HCV Synth + | 16.06 | 0.12 | 0.73 | 3 |
| | 2 μm FIP | | | | |
| | HCV Synth + | 16.07 | 0.26 | 1.59 | 3 |
| | 5 μm FIP | | | | |
| | HCV Synth +10 | 16.08 | 0.18 | 1.11 | 3 |
| | μm FIP | | | | |
| | Avg. NTCs Neat | 42.69 | 1.32 | 3.08 | |
| | NTCs + | 57.51 | 11.54 | 20.07 | 6 |
| | 2 μm FIP | | | | |
| | NTCs + | 74.70 | 22.32 | 29.87 | 6 |
| | 5 μm FIP | | | | |
| | NTCs + | 82.10 | 19.27 | 23.48 | 5 |
| | 10 μm FIP | | | | |
| HAV FIP1_8b | Avg. HCV | 16.51 | 0.18 | 1.08 | |
| ext Phos3′ | Synth Neat | | | | |
| | HCV Synth + | 102.12 | 2.19 | 2.14 | 3 |
| | 2 μm FIP | | | | |
| | HCV Synth + | | | | 0 |
| | 5 μm FIP | | | | |
| | HCV Synth + | | | | 0 |
| | 10 μm FIP | | | | |
| | Avg. NTCs Neat | 42.69 | 1.32 | 3.08 | |
| | NTCs + | | | | 0 |
| | 2 μm FIP | | | | |
| | NTCs + | | | | 0 |
| | 5 μm FIP | | | | |
| | NTCs + | | | | 0 |
| | 10 μm FIP | | | | |
| HAV FIP1_ | Avg. HCV | 16.51 | 0.18 | 1.08 | |
| 16b full | Synth Neat | | | | |
| ext_Phos3′ | HCV Synth + | 21.55 | 0.33 | 1.52 | 3 |
| | 2 μm FIP | | | | |
| | HCV Synth + | 36.23 | 0.14 | 0.38 | 3 |
| | 5 μm FIP | | | | |
| | HCV Synth + | 67.58 | 1.39 | 2.05 | 3 |
| | 10 μm FIP | | | | |
| | Avg. NTCs Neat | 42.69 | 1.32 | 3.08 | |
| | NTCs + | | | | 0 |
| | 2 μm FIP | | | | |
| | NTCs + | | | | 0 |
| | 5 μm FIP | | | | |
| | NTCs + | | | | 0 |
| | 10 μm FIP | | | | |

Unmodified HAVFIP1 linearly slowed the amplification of positives, with 10 μM HAVFIP1 reactions taking approximately 4 times as long to amplify as no-FIP samples. The NTCs were heavily slowed at 2 μM HAVFIP1 and gone entirely at and above 5 HAVFIP1. Phosphorylated HAVFIP1 did not slow the true positives but did inhibit amplification of the false positives. For the HAVFIP1_4b oligonucleotide, a mild slowing of the positives at 2 μM and 5 μM was observed, while adding 10 μM HAVFIP1_4b was approximately equivalent to adding 2 μM of HAVFIP1 in terms of the amplification times for the positives. Amplification of negatives was slowed in all cases. For the HAVFIP1_8b oligonucleotide, no false positive amplification was observed, while amplification of the positives was inhibited above 5 μM HAVFIP1_8b. For the HAVFIP1_12b oligonucleotide, positives were slowed to a much greater degree than unmodified HAVFIP1, and completely suppressed NTCs at all tested concentrations. For the HAVFIP1_16b oligonucleotide, amplification results were very similar to the unmodified HAVFIP1. The lengths of hairpin affected activity of the HAVFIP1 variants. Notably, phosphorylated HAVFIP1 did not slow the true positives, but did inhibit amplification of the false positives. Thus, HAVFIP1 and derivatives of HAVFIP1 suppressed nonspecific amplification in LAMP reactions.

### Example 2—Activity of HAVFIP1 Short Extension Variants

The activity of various HAVFIP1 short extension variants was tested in a LAMP assay with an HCV template and an HCV LAMP primer set. LAMP primer sets are listed in TABLES 14-19. The HAVFIP1 short extension variants included a HAVFIP1 oligonucleotide having a phosphorylated 3′ end; HAVFIP1 oligonucleotides having a phosphorylated 1-, 2-, 3-nucleotide 3′ extension; a HAVFIP1 hairpin variant; a HAVFIP1 early complement variant; and a HAVFIP1 hairpin mirror variant. The variants are listed in TABLE 3.

TABLE 3

| Oligomer | Hairpin Tm (° C.) | SEQ ID NO |
|---|---|---|
| HAVFIP1 Phos3′ | | SEQ ID NO: 01 |
| HAVFIP1_1b ext_Phos3′ | 60.7 | SEQ ID NO: 03 |
| HAVFIP1_2b ext_Phos3′ | 62.4 | SEQ ID NO: 04 |
| HAVFIP1_3b ext_Phos3′ | 63.5 | SEQ ID NO: 05 |
| HAVFIP1_hairpin | 53.7 | SEQ ID NO: 09 |
| HAVFIP1_early complement | 48.1 | SEQ ID NO: 10 |
| HAVFIP1_hairpin mirror | 47.6 | SEQ ID NO: 18 |

Reactions conditions were: 20 mM Tris-HCl (pH 8.8 at 25° C.), 8 mM magnesium sulfate, 50 mM potassium chloride, 10 mM ammonium sulfate, 0.1% Tween-20, 8 U WarmStart Bst 2.0 DNA Polymerase, 7.5 U WarmStart RTx Reverse Transcriptase, 5.6 mM total dNTPs (1.4 mM each), 0.5 U Antarctic Thermolabile UDG, 0.7 mM dUTP, 1× EvaGreen intercalating dye, and primer set “HCV Pr5” (comprised of 40 μmol each FIP/BIP, 10 μmol each LF/LB, and 5 μmol each F3/B3). Positive samples contained the “HCV Synth” mixtures while NTCs contained no templates. Each HAVFIP1 variant from TABLE 3 was present at a concentration of 10 μM. The total reaction volume was 25 μL. Reactions with template were tested in triplicate, and NTCs were tested in 6 replicates. The LAMP reactions were run at 65° C. for 120 minutes on an Applied Biosystems QuantStudio 3 (QS3) thermocycler. LAMP reactions were run at 65° C. for 120 minutes, with fluorescence measured once per minute. Results are summarized in TABLE 4.

TABLE 4

| | Ct | Ct | | |
|---|---|---|---|---|
| Sample Name | Mean | SD | % RSD | Comment |
| Positive Neat | 17.1 | 0.5 | 2.7 | |
| Negative Neat | 46.1 | 11.8 | 25.6 | 6 replicates |
| | | | | amplified |
| Positive + HAV FIP1_Phos3′ | 17.5 | 0.5 | 3.0 | +0.5 CT vs. Neat |
| Negative + HAV FIP1_Phos3′ | no amplifications | | | |
| Positive + HAV FIP1_1b ext_Phos3′ | 18.0 | 0.2 | 1.2 | +1.0 CT vs. Neat |
| Negative + HAV FIP1_1b ext_Phos3′ | 117.8 | | n/a | 1 replicate amplified |
| Positive + HAV FIP1_2b ext_Phos3′ | 18.2 | 0.2 | 1.1 | +1.1 CT vs. Neat |
| Negative + HAV FIP1_2b ext_Phos3′ | no amplifications | | | |
| Positive + HAV FIP1_3b ext_Phos3′ | 18.5 | 0.2 | 0.9 | +1.4 CT vs. Neat |
| Negative + HAV FIP1_3b ext_Phos3′ | 94.6 | 23.0 | 24.3 | 3 replicate |
| | | | | samplified |
| Positive + HAV FIP1_hairpin | 17.8 | 0.3 | 1.8 | +0.7 CT vs. Neat |
| Negative + HAV FIP1_hairpin | 77.0 | 21.2 | 27.5 | 6 replicates |
| | | | | amplified |
| Positive + HAV FIP1_early complement | 50.0 | 0.1 | 0.2 | +32.9 CT vs. Neat |
| Negative + HAV FIP1_early complement | no amplifications | | | |
| Positive + HAV FIP1_hairpin mirror | 17.8 | 0.3 | 1.9 | +0.7 CT vs. Neat |
| Negative + HAV FIP1_hairpin mirror | 33.4 | 4.4 | 13.1 | 6 replicates |
| | | | | amplified |

Each tested HAVFIP1 variant, except HAVFIP1_hairpin mirror, had activity to reduce amplification of false positives. HAVFIP1_Phos3′ completely suppressed false-positive amplification while only mildly slowing the true positives. The base-by-base extensions of HAVFIP1 slowed true positives more and more as bases were added. The HAVFIP1_3b ext_Phos3′ variant allowed three false positives to amplify, albeit with an average Ct of about 95 min. The HAVFIP1_hairpin variant which included only a hairpin-forming sequence only slowed LAMP more than the HAVFIP1_Phos3′ variant. The HAVFIP1_hairpin variant slowed the negatives to a statistically significant degree vs. the Neat negatives (46.1±11.8 min vs. 77.0±21.2 min for Neat and Hairpin, respectively; p=0.014 for two-tail t-test). This showed that the hairpin segment is sufficient to have activity to suppress false positive amplification. The HAVFIP1 early complement variant which conserves the hairpin sequence and flips all of the preceding bases to their complements, slowed the amplification of true positives by a factor of nearly 3 (17.1 min vs. 50.0 min), and also completely eliminated false-positive amplification. On the other hand, native HAVFIP1 slowed LAMP by about 25% with HCV Pr5. HAVFIP1_hairpin mirror, in which the bases were complementary to HAVFIP1_hairpin, albeit not reverse complementary, increases false-positive amplification. The HAVFIP1_hairpin mirror mildly slowed true positives while accelerating false-positive amplification (33.4±4.4 min vs. 46.1±11.8 min for hairpin mirror and Neat, respectively; two-tail p=0.048). Thus, the hairpin region alone had activity to suppress false-positive amplification. Adding the HAVFIP1 stem increased the suppression of false-positive amplification, and flipping the stem to its complement made the sequence more inhibitory to LAMP.

### Example 3—Effects of HAVFIP1 Concentration

LAMP reactions were performed with varying concentrations of phosphorylated HAVFIP1 (SEQ ID NO:01). Reactions included HCV targets, HCV LAMP primer sets, and various concentrations of phosphorylated HAVFIP1. Reactions were prepared using a LAMP WARMSTART Master Mix (New England Biolabs, Ipswich, Mass.) which contained a blend of Bst 2.0 WARMSTART DNA Polymerase and WARMSTART RTx Reverse Transcriptase in an optimized LAMP buffer solution. LAMP primer sets are listed in TABLES 14-19. All reactions were performed in triplicate. Positive samples (“SC P HCV 2” and “DLS HCV 6”) were HCV-infected human plasma, of which 1.25 μL were added to each reaction. LAMP reactions were run on a QS3 at 65° C. for 120 minutes on a QS3 thermocycler. Fluorescence was measured once per minute to assess reaction progress. TABLE 5 lists reaction components and summarizes the results.

TABLE 5

| | Inhibitory | LAMP | Ct Mean | Ct SD | |
|---|---|---|---|---|---|
| Target | oligonucleotide | primer set | (min) | (min) | % RSD |
| SC P HCV 2 | 0 μM phospho- | HCV O Pr 1 | 22.06 | 0.66 | 2.99 |
| | HAVFIP1 | | | | |
| Absent | 0 μM phospho- | HCV O Pr 1 | 47.94 | 7.71 | 16.09 |
| | HAVFIP1 | | | | |
| SC P HCV 2 | 2 μM phospho- | HCV O Pr 1 | 22.52 | 0.81 | 3.60 |
| | HAVFIP1 | | | | |
| Absent | 2 μM phospho- | HCV O Pr 1 | 62.20 | 12.94 | 20.80 |
| | HAVFIP1 | | | | |
| SC P HCV 2 | 10 μM phospho- | HCV O Pr 1 | 26.16 | 0.52 | 1.99 |
| | HAVFIP1 | | | | |
| Absent | 10 μM phospho- | HCV O Pr 1 | 86.06 | — | — |
| | HAVFIP1 | | | | |
| DLS HCV6 | 0 μM phospho- | HCV Pr 5 | 11.19 | 0.07 | 0.59 |
| | HAVFIP1 | | | | |
| Absent | 0 μM phospho- | HCV Pr 5 | 48.78 | 5.31 | 10.88 |
| | HAVFIP1 | | | | |
| DLS HCV6 | 1 μM phospho- | HCV Pr 5 | 11.23 | 0.07 | 0.64 |
| | HAVFIP1 | | | | |
| Absent | 10 μM phospho- | HCV Pr 5 | 54.10 | 8.45 | 15.62 |
| | HAVFIP1 | | | | |
| DLS HCV6 | 2 μM phospho- | HCV Pr 5 | 11.58 | 0.03 | 0.27 |
| | HAVFIP1 | | | | |
| Absent | 2 μM phospho- | HCV Pr 5 | 65.12 | 14.74 | 22.63 |
| | HAVFIP1 | | | | |
| DLS HCV6 | 5 μM phospho- | HCV Pr 5 | 11.76 | 0.16 | 1.33 |
| | HAVFIP1 | | | | |
| Absent | 5 μM phospho- | HCV Pr 5 | 105.65 | 11.00 | 10.41 |
| | HAVFIP1 | | | | |
| DLS HCV6 | 10 μM phospho- | HCV Pr 5 | 12.44 | 0.05 | 0.37 |
| | HAVFIP1 | | | | |
| Absent | 10 μM phospho- | HCV Pr 5 | — | — | — |
| | HAVFIP1 | | | | |

A high concentration of 10 μM phospho-HAV FIP did not affect the amplification of the clinical sample of HCV but did completely inhibit the amplification of no template control (NTC) reactions in which the target was absent. NTC Ct values increased as the concentration of the phospho-HAV FIP increased. The presence of phospho-HAVFIP1 had no substantial effect on the HCV LAMP reaction with target present. However, in NTC reactions, the presence of phospho-HAVHIP1 in the HCV LAMP reaction increased the Ct value, and the Ct value increased with an increase in the phospho-HAVFIP1 concentration. Thus, phosphorylated HAVFIP1 inhibited nonspecific amplification in the LAMP reactions in a concentration dependent manner.

### Example 4—Activity of Alternative Inhibitory Oligomers in LAMP

The activity of various oligonucleotides to inhibit nonspecific amplification in a LAMP reaction inhibitory was tested. Reactions contained LAMP HCV primers (HCV Pr5), and various test oligonucleotides, and no target nucleic acid. Reactions were prepared with a LAMP WARMSTART Master Mix (New England Biolabs, Ipswich, Mass.), which contained a blend of Bst 2.0 WARMSTART DNA Polymerase and WARMSTART RTx Reverse Transcriptase in an optimized LAMP buffer solution. Test oligonucleotides were tested at 2 μM. LAMP primer sets are listed in TABLES 14-19. Reactions were performed in six replicates. Neat samples contained no test oligonucleotide. LAMP reactions were run at 65° C. for 120 minutes on a QS3 thermocycler. Fluorescence was measured once per minute to assess reaction progress. TABLE 6 lists test oligonucleotides. TABLE 7 summarizes the results.

TABLE 6

| Oligonucleotide | Tm (° C.) | SEQ ID NO |
|---|---|---|
| HAVF IP1 | 53.7 | SEQ ID NO: 01 |
| HAVB IP1 | 60.6 | SEQ ID NO: 97 |
| Dev3 BIP | 52.7 | SEQ ID NO: 15 |
| Dev3 BIP_4b ext | 64.8 | SEQ ID NO: 11 |
| Dev3 BIP_17b full ext | 92 | SEQ ID NO: 12 |
| HAVFIP1_2b cut | 47.1 | SEQ ID NO: 17 |
| HAVFIP1_4b cut | 43.1 | SEQ ID NO: 13 |
| RSV Pr1 FTP | 50.6 | SEQ ID NO: 85 |
| RSV Pr2 FIP | 53.2 | SEQ ID NO: 14 |
| TB Pr2 BIP | 50.5 | SEQ ID NO: 16 |
| TB Pr3 BIP | 53.2 | SEQ ID NO: 111 |

TABLE 7

| | Ct | Ct | % | # amplified | ΔCt vs. | Two- |
|---|---|---|---|---|---|---|
| Sample | mean | SD | RSD | replicates | neat | tailed p |
| Neat | 44.9 | 3.3 | 7.3 | 6 | n/a | |
| HAVFIP1 | 75.7 | | n/a | 1 | +30.7 | strong |
| HAVBIP1 | 41.1 | 3.3 | 8.0 | 6 | -3.8 | >0.05 |
| Dev3 BIP | 61.0 | 6.2 | 10.1 | 6 | +16.1 | <0.05 |
| Dev3 BIP_4b ext | 70.0 | | n/a | 1 | +25.1 | strong |
| Dev3 BIP_17b full ext | No amplifications | | | | | strong |
| HAVFIP1_2b cut | 47.7 | 18.5 | 38.8 | 5 | +2.8 | >0.05 |
| HAVFIP1_4b cut | 92.6 | 6.4 | 6.9 | 4 | +47.6 | <0.01 |
| RSV Pr1 FTP | 49.0 | 13.6 | 27.6 | 6 | +4.1 | >0.05 |
| RSV Pr2 FTP | 106.5 | | n/a | 1 | +61.5 | strong |
| TB Pr2 BIP | 47.4 | 8.9 | 18.8 | 6 | +2.5 | >0.05 |
| TB Pr3 BIP | 52.9 | 4.0 | 7.7 | 6 | +7.9 | <0.01 |

ΔCt vs. Neat: relates to the difference between the average Ct for that condition and the average Ct for the Neat samples (negative controls), with positive being slower than Neat and negative being faster.
Two-tail p: relates to the results of a two-tailed t-test between that sample and Neat. Those with p-values > 0.05 are not different from Neat to a statistically significant degree. Those with p-values < 0.05 have reached statistical significance. Those simply labeled “Strong” had extremely significant effects (no amplified replicates, or few/very late amplifications) and were not t-tested.

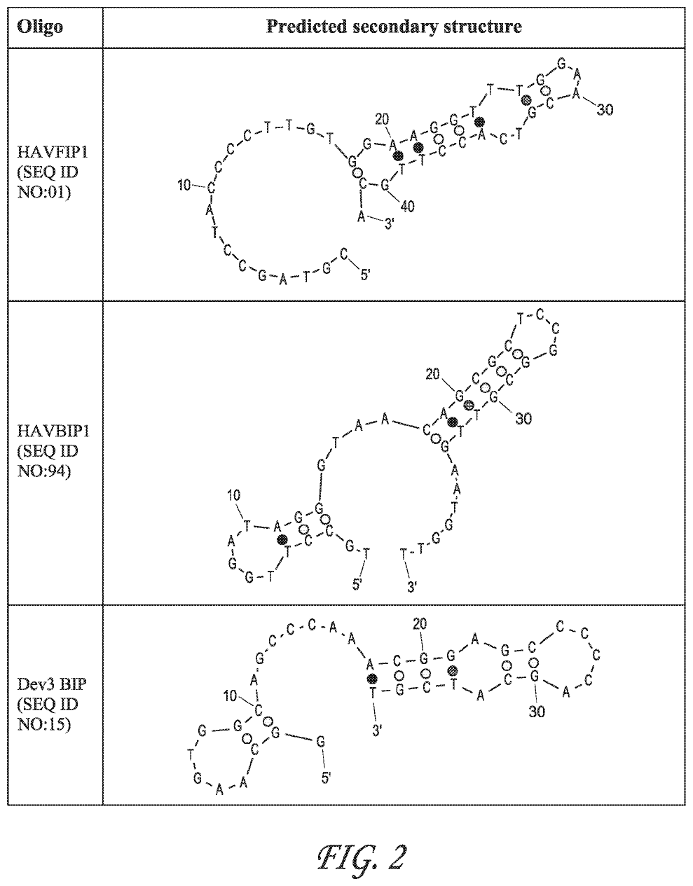

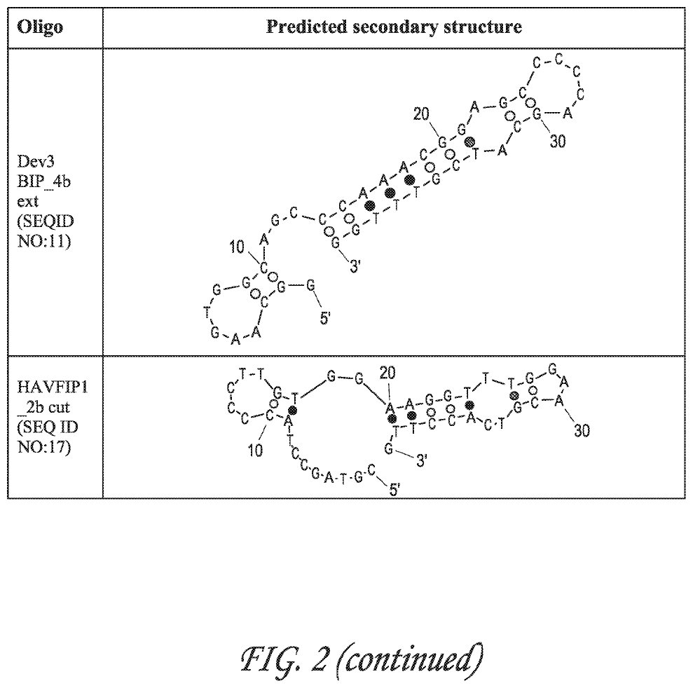

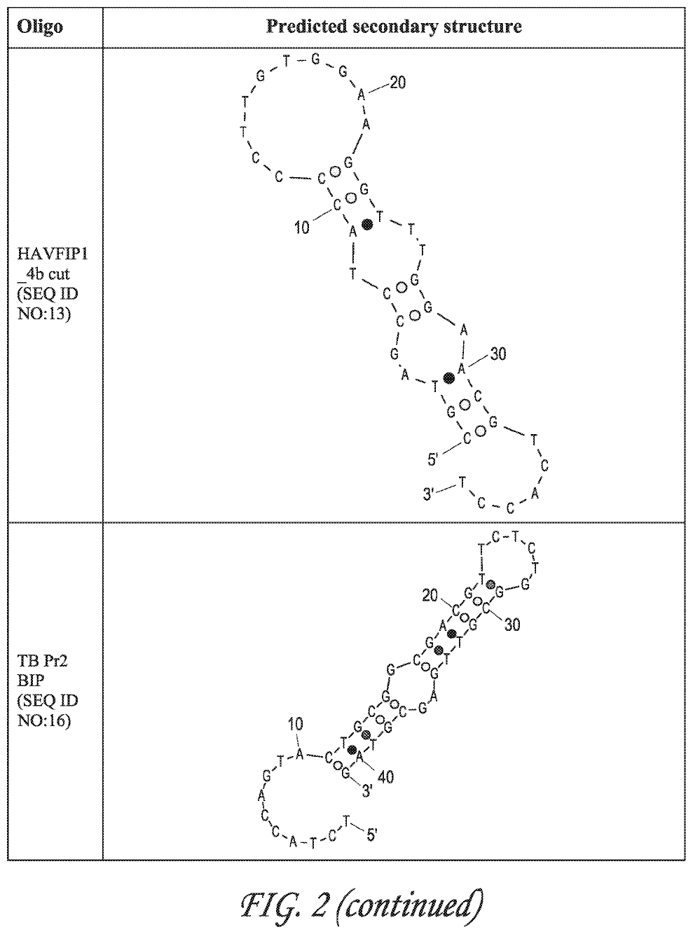

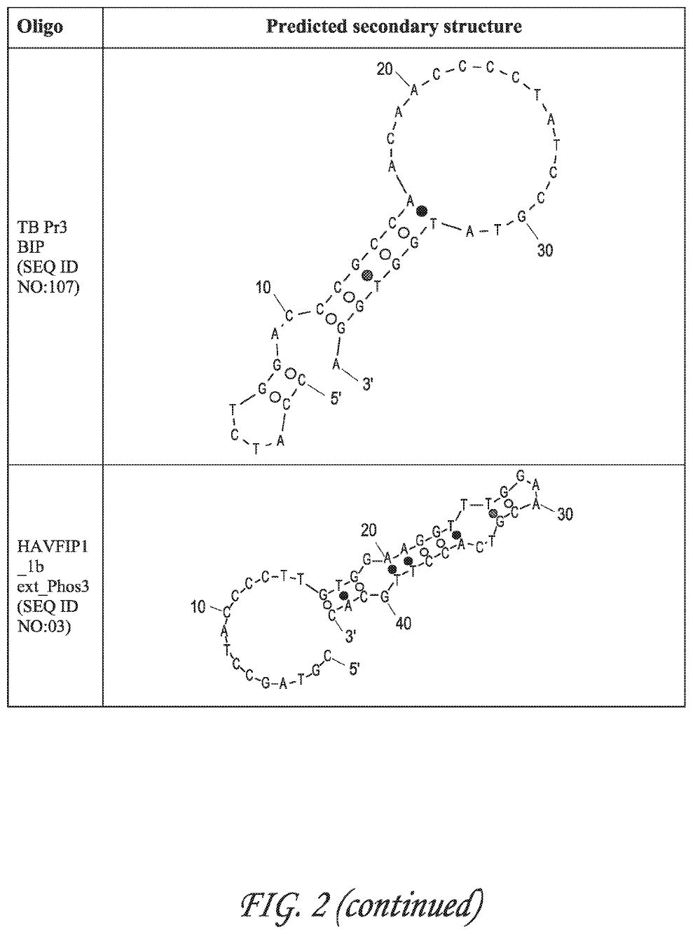

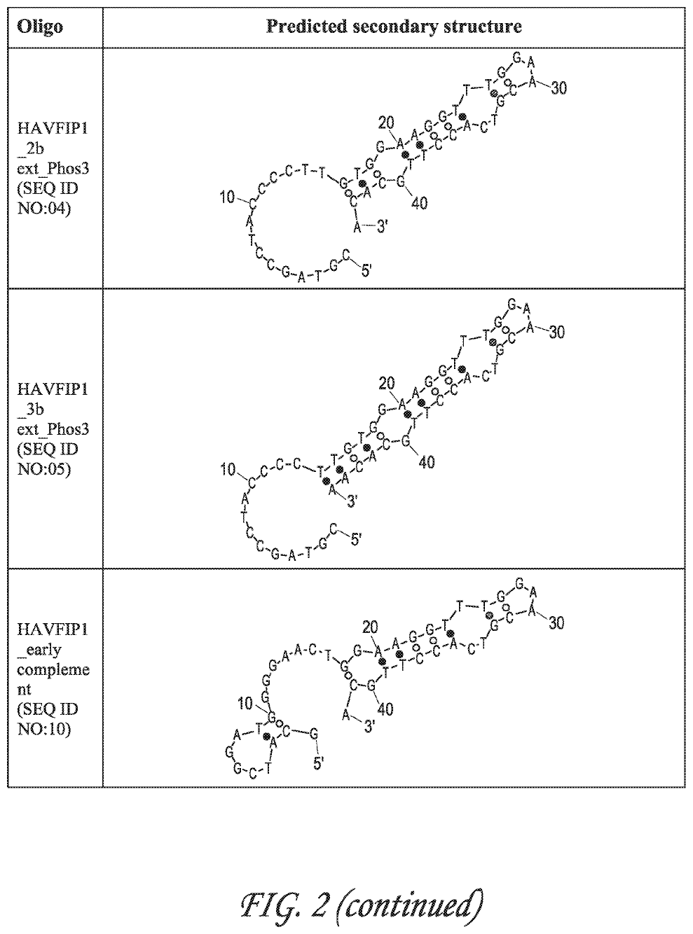

The predicted secondary structures of certain oligonucleotides are shown in FIG. 2. The following oligomers had strong activity, similar to HAVFIP1, to suppress false positives in no template controls in a LAMP amplification reaction: Dev3 BIP_4b ext, Dev3 BIP_17b full ext, and RSV Pr2 FIP. The following oligomers had some activity to suppress false positives in no template controls in a LAMP amplification reaction compared to negative control (Neat), however nonspecific amplification of false positives was not eliminated or dramatically slowed: Dev3 BIP, HAVFIP1_4b cut, and TB Pr3 BIP. The following oligomers had no substantial activity to suppress false positives in no template controls in a LAMP amplification reaction and were comparable to the negative control (Neat): HAV BIP1, HAVFIP1_2b cut, and TB Pr2 BIP.

### Example 5—Activity of HAVFIP1 Variants with Certain LAMP Primer Sets

The activity of a HAVFIP1 variant mixture containing a HAVFIP1 oligonucleotide having a phosphorylated 3′ end (SEQ ID NO:01), and a HAVFIP1 oligonucleotide variant having a phosphorylated 4-nucleotide 3′ extension (SEQ ID NO:02) was tested in a LAMP assay with various LAMP primer sets. LAMP primer sets included: Dengue Pr1; Dengue Pr2; HCV Pr4; HCV Pr6; Zika Pr1; and Zika Pr3. LAMP primer sets are listed in TABLES 14-19. Each of the LAMP primer sets had demonstrated NTC amplification at early times (˜30-40 min) during LAMP reactions. WarmStart LAMP Master Mix was used to prepare 8 replicates of all samples. LAMP reactions were run at 65° C. for 120 minutes on a QS3 thermocycler. Fluorescence was measured once per minute to assess reaction progress. The HAVFIP1 variant mixture (“FLASH”) was made including 10 μM HAVFIP1_4b ext_Phos3′ and 50 μM HAVFIP1_Phos3′, and this was added to reactions such that their final concentrations were 2 μM and 10 respectively. No reactions included a template. Results are summarized in TABLE 8.

TABLE 8

| | Ct | Ct | | Earliest | # Amplified |
|---|---|---|---|---|---|
| Sample | Mean | SD | % RS | Amp (Ct) | Replicates |
| Dengue Pr1 Neat | 31.40 | 2.08 | 6.64 | 29.35 | 8 |
| Dengue Pr1 + FLASH | 105.85 | 16.92 | 15.98 | 93.88 | 2 |
| Dengue Pr2 Neat | 36.72 | 10.40 | 28.32 | 30.85 | 8 |
| Dengue Pr2 + FLASH | 84.21 | 20.66 | 24.53 | 60.46 | 5 |
| HCV Pr4 Neat | 54.83 | 8.34 | 15.21 | 44.28 | 8 |
| HCV Pr4 + FLASH | no amplifications | | | | |
| HCV Pr6 Neat | 76.76 | 10.88 | 14.18 | 69.07 | 2 |
| HCV Pr6 + FLASH | no amplifications | | | | |
| Zika Pr1 Neat | 48.06 | 3.27 | 6.81 | 41.63 | 8 |
| Zika Pr1 + FLASH | 77.90 | 13.56 | 17.40 | 58.63 | 5 |
| Zika Pr3 Neat | 77.85 | 22.29 | 28.63 | 55.88 | 5 |

| | |
|---|---|
| Zika Pr3 + FLASH | no amplifications |

The HAVFIP1 variant mixture had a marked effect on all reactions containing LAMP primer sets. All eight replicates of HCV Pr4 Neat amplified with an average Ct of ˜55 min, but zero amplified with the HAVFIP1 variant mixture. The HAVFIP1 variant mixture also prevented any replicates of Zika Pr3 and HCV Pr6 from amplifying. The HAVFIP1 variant mixture had reduced effects on LAMP reactions containing the Zika Pr1 LAMP primer set (Avg. Ct ˜48 min Neat, 78 min with the HAVFIP1 variant mixture), and on LAMP reactions contain the Dengue Pr2 LAMP primer set (Avt. Ct ˜37 min Neat, ˜84 min with the HAVFIP1 variant mixture).

### Example 6—Amplification of Targets with Certain LAMP Primer Sets

This example illustrates LAMP amplification of targets with a mixture of 15 LAMP primer sets containing a LAMP primer set against a target. Each mixture contained HAVFIP1. Targets included Synt RSV, Synt Zika, Vircell Dengue, Synt HAV, Synt HBV, Synt HCV mix, Synt HIV mix, Synt Parvo, ATCC FluA, ATCC Sal, Syth TB_3, Synt H inf, Synt Dev2, MS2, Synt Malaria. Fifteen LAMP primer sets were mixed together and included RSV primer_new LB, Zika_2 primer, Dengue_1 primer, HAV primers, HBV primers, HCV pr 5, HIV primer 1, Parvo primers, Sal_2 primers, TB_3 primers, H inf primers, Dev2 primers, MS2 primers, Malaria primers made 180123, FluAH3N1_5 primers. See e.g., Kim DW, et al., J Clin Microbiol (2011) 49:3621-3626; and Chander Y, et al., Front Microbiol (2014) 5:395, which are each incorporated by reference in its entirety. LAMP primer sets are listed in TABLES 14-19. HAVFIP1 (SEQ ID NO:01) was present in the experiment in the HAV primer mix at a concentration of 1.6 μM.

Reactions were prepared by adding 3 μL per reaction of the 15-primer mix to a master mix. Using WarmStart Master Mix, 4 replicates of each individual target were made in each tube. In other words, each tube had a different target in it. 8 NTC reactions were also made. Each synthetic reaction has 10⁶ copies of target. The ATCC Sal reactions contained 4.8×10⁵ copies, and the ATCC FluA (H3N2) samples contained 4.24×10⁷ copies of target. LAMP reactions were run at 65° C. for 120 minutes on a QS3 thermocycler. Fluorescence was measured once per each minute to assess reaction progress. TABLE 9 summarizes the results.

TABLE 9

| Sample | Ct Mean | Ct SD | % RSD |
|---|---|---|---|
| Synt RSV | 28.88 | 0.17 | 0.58 |
| Synt Zika | 19.00 | 0.06 | 0.31 |
| Vircell Dengue | — | — | — |
| Synt HAV | 44.00 | 0.24 | 0.55 |
| Synt HBV | 27.32 | 0.28 | 1.04 |
| Synt HCV | 29.28 | 0.36 | 1.22 |
| Synt HIV | 30.71 | 0.05 | 0.16 |
| Synt Parvo | 39.89 | 0.55 | 1.37 |
| ATCC Sal | 44.83 | 0.28 | 0.62 |
| Synt TB | 30.52 | 0.05 | 0.15 |
| Synt H Inf | 57.38 | 0.18 | 0.32 |
| Synt Dev2 | 30.01 | 0.52 | 1.74 |
| MS2 | 17.38 | 0.19 | 1.08 |
| Synt Malaria | 41.10 | 0.09 | 0.23 |
| ATCC FluA | 65.85 | 0.39 | 0.59 |
| NTC with all primers | — | — | — |

All of the positive reactions amplified except for Vircell Dengue with the 15-primer mix. None of the NTC reactions amplified. This indicates that it is possible to perform highly multiplexed and specific LAMP if HAVFIP1 is included in the reaction.

### Example 7—Use of PEG to Enhance LAMP

LAMP was tested with and without additional Bst 2.0 and 5% polyethylene glycol-35k (PEG) in order to improve sensitivity and overall time to result. In prior experiments, replicates containing 5% PEG amplified earlier than those not containing PEG. Reactions were based on WarmStart LAMP Master Mix and all contained RSV A_B 4 primers. Test conditions were: LAMP Mix with 5% PEG and an additional 3 (+3 Bst 2.0; LAMP Mix with 5% PEG and +0 μL Bst 2.0; LAMP Mix with 0% PEG and +3 μL Bst 2.0; and LAMP Mix with 0% PEG and +0 μL Bst 2.0 (LAMP Control). LAMP primer sets are listed in TABLES 14-19. RSV AB Megamer was included as a template at 10⁶, 10⁴, 10², 1, and 0 (NTCs) copies per reaction (C/rxn). LAMP reactions were run at 65° C. for 120 minutes on a QS3 thermocycler. Fluorescence was measured once per minute to assess reaction progress. Results are summarized in TABLE 10.

TABLE 10

| | RSV AB | | | | |
|---|---|---|---|---|---|
| | Megamer | Ct | Ct | | # |
| | Concentration | Mean | SD | % | Replicates |
| Additives | (C/rxn) | (min) | (min) | RSD | Amplified |
| 5% PEG; + | 10⁶ | 7.28 | 0.09 | 1.30 | 4/4 |
| 3 μL | 10⁴ | 9.45 | 0.23 | 2.47 | 4/4 |
| Bst 2.0 | 10² | 30.53 | 20.12 | 65.89 | 4/4 |
| | 1 | 50.98 | 10.00 | 19.61 | 4/4 |
| | NTC | 46.54 | 12.83 | 27.56 | 4/4 |
| 0% PEG; + | 10⁶ | 8.86 | 0.06 | 0.63 | 4/4 |
| 3 μL | 10⁴ | 10.81 | 0.17 | 1.55 | 4/4 |
| Bst 2.0 | 10² | 40.19 | 24.72 | 61.49 | 4/4 |
| | 1 | 61.30 | 21.89 | 35.71 | 3/4 |
| | NTC | 77.17 | 2.31 | 2.99 | 2/4 |
| 5% PEG; + | 10⁶ | 7.86 | 0.05 | 0.62 | 4/4 |
| 0 μL | 10⁴ | 9.82 | 0.29 | 2.96 | 4/4 |
| Bst 2.0 | 10² | 21.05 | 14.58 | 69.27 | 4/4 |
| | 1 | 35.58 | 5.79 | 16.28 | 4/4 |
| | NTC | 60.33 | 17.18 | 28.47 | 4/4 |
| 0% PEG; + | 10⁶ | 13.53 | 0.11 | 0.81 | 4/4 |
| 0 μL | 10⁴ | 16.36 | 0.28 | 1.69 | 4/4 |
| Bst 2.0 | 10² | 34.65 | 15.91 | 45.92 | 4/4 |
| (LAMP | 1 | 45.17 | 8.54 | 18.90 | 4/4 |
| CTRL) | NTC | 46.86 | 3.28 | 7.00 | 4/4 |

All values were evaluated with Grubb's test to determine whether there was an outlier. For the ones that had an outlier, it was marked with a (*) and the average Ct and standard deviations were recalculated without the outlier. Adding 3 μL of Bst 2.0 decreased Cts but did not improve the detection of lower copy numbers. Replicates containing 5% PEG and +3 μL Bst 2.0 had the earliest Cts overall, however were only slightly faster than those containing just 5% PEG.

### Example 8—Use of Different Crowding Agents to Enhance LAMP

A dynamic range of RSV AB Megamer was tested with various crowding agents to determine whether lower copy numbers could be detected. The following conditions were tested: 5% PEG-35K; 5% PEG-8K; 5% Ficoll-400K. Each condition was tested with serial 10-fold dilutions of RSV AB Megamer from 10⁶ to 0 copies/reaction. 3 replicates of each condition were tested. LAMP primer sets are listed in TABLES 14-19. LAMP reactions were run at 65° C. for 120 minutes on a QS3 thermocycler. Fluorescence was measured once per minute to assess reaction progress. TABLE 11 summarizes the results.

TABLE 11

| | Concentration | | | | |
|---|---|---|---|---|---|
| | RSV AB | Ct | Ct | | # |
| Crowding | Megamer | Mean | SD | % | Replicates |
| Agent | (C/rxn) | (min) | (min) | RSD | Amplified |
| 5% PEG- | 10⁶ | 8.895 | 0.007 | 0.079 | 3/3 |
| 35K | 10⁵ | 10.211 | 0.272 | 2.665 | 3/3 |
| | 10⁴ | 12.600 | 0.484 | 3.845 | 3/3 |
| | 10³ | 15.538 | 1.509 | 9.710 | 3/3 |
| | 10² | 25.619 | 9.875 | 38.546 | 3/3 |
| | 10 | 25.535 | 1.468 | 5.749 | 3/3 |
| | 1* | 46.293 | 0.129 | 0.278 | 3/3 |
| | NTC | 37.918 | 8.597 | 22.673 | 3/3 |
| 5% PEG- | 10⁶ | 8.094 | 0.072 | 0.893 | 3/3 |
| 8K | 10⁵ | 9.642 | 0.071 | 0.734 | 3/3 |
| | 10⁴ | 11.479 | 0.220 | 1.916 | 3/3 |
| | 10³ | 15.326 | 2.981 | 19.449 | 3/3 |
| | 10² | 23.416 | 8.041 | 34.340 | 3/3 |
| | 10 | 30.666 | 1.433 | 4.672 | 3/3 |
| | 1 | 38.658 | 5.012 | 12.965 | 3/3 |
| | NTC | 32.033 | 1.490 | 4.651 | 2/3 |
| 5% Ficoll- | 10⁶ | 10.015 | 0.110 | 1.096 | 3/3 |
| 400K | 10⁵ | 11.748 | 0.066 | 0.562 | 3/3 |
| | 10⁴ | 13.431 | 0.376 | 2.803 | 3/3 |
| | 10³ | 16.152 | 2.494 | 15.442 | 3/3 |
| | 10² | 18.180 | 2.986 | 16.423 | 2/3 |

| | | |
|---|---|---|
| 10 | No Amplification | 0/3 |
| 1 | No Amplification | 0/3 |
| NTC | No Amplification | 0/3 |

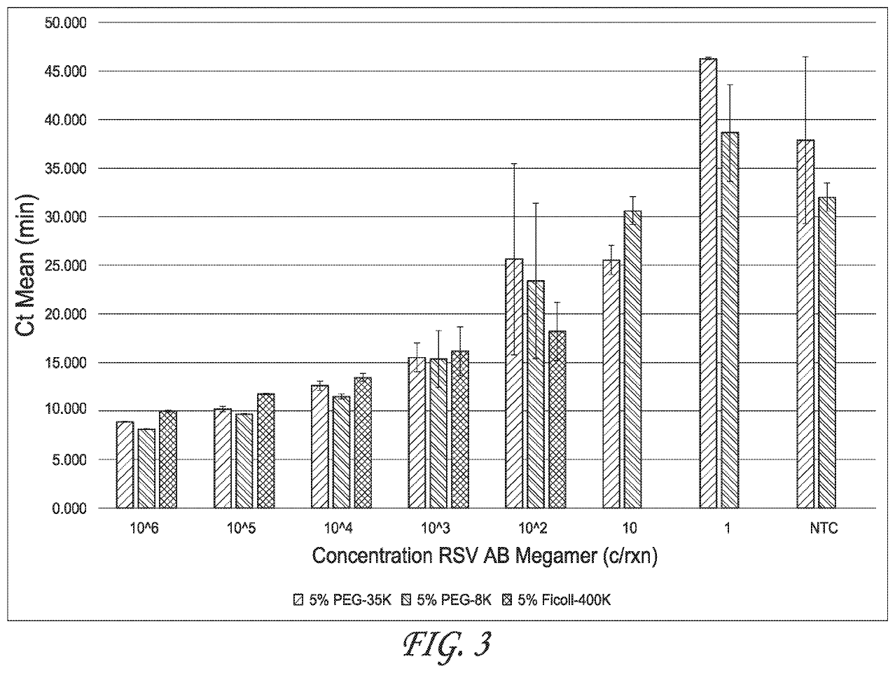

All values were evaluated with Grubb's test to determine whether there is an outlier. For the ones that had an outlier, it was marked with a (*) and the average Ct and standard deviations were recalculated without the outlier. FIG. 3 is a graph which summarizes Ct values for different concentrations of target for reactions containing different crowding agents. 5% PEG-8K had the earliest Cts overall, though the three lowest concentrations overlapped in amplification with the NTCs. While 5% Ficoll-400K did not have any NTCs, there was also no detection of 10 or 1 copies per reaction.

### Example 9—LAMP with Inhibitory Oligonucleotides and a Crowding Agent

The effect of a mixture of inhibitory oligonucleotides (Phospho-FIP/Phospho-FIP4) with PEG-35K on various targets were evaluated with and without Universal Transport Media (UTM) present in the sample. The inhibitory oligonucleotide mixture was a HAVFIP1 variant mixture containing a HAVFIP1 oligonucleotide having a phosphorylated 3′ end (Phospho-FIP; SEQ ID NO:01), and a HAVFIP1 oligonucleotide variant having a phosphorylated 4-nucleotide 3′ extension (Phospho-FIP4, SEQ ID NO:02). Targets included: a synthetic (Synt) HIV 1C, Synt HCV 1, RSV AB Megamer, FluA_M2_180815_2. LAMP primer sets included: HIV 1 Primers, HCV 10 Primers, RSV A_B 4 Primers, FluA_180817_H3N2_2/3. Example LAMP primer sets are listed in TABLES 14-19.

The following conditions were tested: Synt HCV 1 with HAVFIP1 variants; Synt HCV 1 without HAVFIP1 variants; FluA_M2_180815_2 with HAVFIP1 variants, no UTM; FluA_M2_180815_2 without HAVFIP1 variants, no UTM; FluA_M2_180815_2 with HAVFIP1 variants and UTM; FluA_M2_180815_2 without HAVFIP1 variants, with UTM; RSV AB Megamer with HAVFIP1 variants, no UTM; RSV AB Megamer without HAVFIP1 variants, no UTM; RSV AB Megamer with HAVFIP1 variants and UTM; RSV AB Megamer without HAVFIP1 variants, with UTM; Synt HIV 1C with HAVFIP1 variants; and Synt HIV 1C without HAVFIP1 variants. Using WarmStart LAMP Master Mix, each condition was tested with 10⁴ copies/reaction of target and NTCs. 3 replicates tested for each condition. HAVFIP1 variants concentration: 10 μM Phospho-FIP; 1 μM Phospho-FIP 4. LAMP reactions were run at 65° C. for 120 minutes on a QS3 thermocycler. Fluorescence was measured once per minute to assess reaction progress. Results are summarized in TABLE 12A.

TABLE 12A

| | | Target | Ct | Ct | | # |
|---|---|---|---|---|---|---|
| | HAVFIP1 | Concentration | Mean | SD | % | Replicates |
| Primers | variants | (c/rxn) | (min) | (min) | RSD | Amplified |
| HCV | Present | 10⁴ | 18.67 | 0.79 | 4.25 | 3/3 |
| | | NTC | 28.80 | 3.22 | 11.19 | 3/3 |
| | Absent | 10⁴ | 17.04 | 1.67 | 9.78 | 3/3 |
| | | NTC | 20.90 | 0.56 | 2.69 | 3/3 |
| FluA | Present | 10⁴ | 31.49 | 0.09 | 0.27 | 3/3 |
| without | | NTC | 47.38 | | | 1/3 |
| UTM | Absent | 10⁴ | 27.44 | 1.95 | 7.10 | 3/3 |
| | | NTC | 47.88 | 5.93 | 12.38 | 3/3 |
| FluA | Present | 10⁴ | 34.10 | 0.49 | 1.44 | 3/3 |
| with | | NTC | No Amplification | | | 0/3 |
| UTM | Absent | 10⁴ | 25.06 | 3.06 | 12.23 | 3/3 |
| | | NTC | 47.11 | 13.22 | 28.06 | 1/3 |
| RSV | Present | 10⁴ | 12.85 | 0.40 | 3.10 | 3/3 |
| without | | NTC | No Amplification | | | 0/3 |
| UTM | Absent | 10⁴ | 11.23 | 0.26 | 2.36 | 3/3 |
| | | NTC | 45.38 | 5.70 | 12.57 | 3/3 |
| RSV | Present | 10⁴ | 13.84 | 0.10 | 0.72 | 3/3 |
| with | | NTC | No Amplification | | | 0/3 |
| UTM | Absent | 10⁴ | 11.80 | 0.23 | 1.99 | 3/3 |
| | | NTC | 53.27 | 4.77 | 8.95 | 2/3 |
| HIV | Present | 10⁴ | 53.49 | | | 1/3 |
| | | NTC | 56.65 | | | 1/3 |
| | Absent | 10⁴ | 25.35 | 1.99 | 7.84 | 3/3 |
| | | NTC | 26.68 | 2.24 | 8.39 | 3/3 |

When used in conjunction with PEG-35K, a mixture of inhibitory oligonucleotides (Phospho-FIP/Phospho-FIP4) was effective at suppressing NTCs.

### Certain Sequences

The following TABLEs list various sequences. TABLE 13 lists various oligonucleotides tested for activity to inhibit nonspecific amplification during a LAMP amplification reaction. TABLES 14-19 lists sets of LAMP primers to detect nucleic acids from certain listed pathogens, including the F3, B3, FIP, BIP, LF and LB for each set. Specifically, TABLE 14 lists F3 primers for each set; TABLE 15 lists B3 primers for each; TABLE 16 lists FIP primers for each set; TABLE 17 lists BIP primers for each set; TABLE 18 lists LF primers for each set; and TABLE 19 lists LB primers for each set.

TABLE 13

| Oligonucleotide | SEQ ID NO | Sequence |
|---|---|---|
| HAVFIP1 | SEQ ID NO: 01 | CGTAGCCTACCCCTTGTGGAAGGTTTGGAA |
| | | CGTCACCTTGCA |
| # HAVFIP1_4b | SEQ ID NO: 02 | CGTAGCCTACCCCTTGTGGAAGGTTTGGAA |
| ext_Phos3′ (aka | | CGTCACCTTGCACAAG |
| Phospho-FIP4) | | |
| # HAVFIP1_1b | SEQ ID NO: 03 | CGTAGCCTACCCCTTGTGGAAGGTTTGGAA |
| ext_Phos3′ | | CGTCACCTTGCAC |
| # HAVFIP1_2b | SEQ ID NO: 04 | CGTAGCCTACCCCTTGTGGAAGGTTTGGAA |
| ext_Phos3′ | | CGTCACCTTGCACA |
| # HAVFIP1_3b | SEQ ID NO: 05 | CGTAGCCTACCCCTTGTGGAAGGTTTGGAA |
| ext_Phos3′ | | CGTCACCTTGCACAA |
| # HAVFIP1_8b | SEQ ID NO: 06 | CGTAGCCTACCCCTTGTGGAAGGTTTGGAA |
| ext_Phos3′ (aka | | CGTCACCTTGCACAAGGGGT |
| Phospho-FIP8) | | |
| # HAVFIP1_12b | SEQ ID NO: 07 | CGTAGCCTACCCCTTGTGGAAGGTTTGGAA |
| ext_Phos3′ (aka | | CGTCACCTTGCACAAGGGGTAGGC |
| Phospho-FIP12) | | |
| # HAVFIP1_16b | SEQ ID NO: 08 | CGTAGCCTACCCCTTGTGGAAGGTTTGGAA |
| full_ext Phos3′ | | CGTCACCTTGCACAAGGGGTAGGCTACG |
| (aka Phospho- | | |
| FIP16) | | |
| HAVFIP1_hairpin | SEQ ID NO: 09 | TGGAAGGTTTGGAACGTCACCTTGCA |
| HAVFIP1_early | SEQ ID NO: 10 | GCATCGGATGGGGAACTGGAAGGTTTGGA |
| complement | | ACGTCACCTTGCA |
| Dev3 BIP_4b ext | SEQ ID NO: 11 | GGCAAGTGGCAGCCCAAACGGAGCCCCCA |
| | | GCATCGTTTGG |
| Dev3 BIP_17b full | SEQ ID NO: 12 | GGCAAGTGGCAGCCCAAACGGAGCCCCCA |
| ext | | GCATCGTTTGGGCTGCCACTTGCC |
| HAVFIP1_4b cut | SEQ ID NO: 13 | CGTAGCCTACCCCTTGTGGAAGGTTTGGAA |
| | | CGTCACCT |
| RSV Pr2 FIP | SEQ ID NO: 14 | TAATGATGCTTTTGGGTTGTTCAATGTTTAT |
| | | GAATATGCCCAAAAATTGG |
| Dev3 BIP | SEQ ID NO: 15 | GGCAAGTGGCAGCCCAAACGGAGCCCCCA |
| | | GCATCGT |
| TB Pr2 BIP | SEQ ID NO: 16 | TCTACCAGTACTGCGGCGACGTTCTCTGGC |
| | | GTTGAGCGTAG |
| HAVFIP1_2b cut | SEQ ID NO: 17 | CGTAGCCTACCCCTTGTGGAAGGTTTGGAA |
| | | CGTCACCTTG |
| HAVFIP1_hairpin | SEQ ID NO: 18 | ACCTTCCAAACCTTGCAGTGGAACGT |
| mirror | | |

# oligomer phosphorylated at 3′ end

TABLE 14
F3 primers

| Primer set (pathogen) | SEQ ID NO | Sequence |
|---|---|---|
| Dengue Pr1 (Dengue virus) | SEQ ID NO: 19 | TCTCTGATGAACAACCAACG |
| Dengue Pr2 (Dengue virus) | SEQ ID NO: 20 | GACCGTCTTTCAATATGCTG |
| Dev2 primers (artificial) | SEQ ID NO: 21 | CGTGTTTGCTCTCACGAA |
| FluAH3N1_5 primers | SEQ ID NO: 22 | CTATCRTCCCGTCAGGC |
| (Influenza A virus, strain | | |
| H3N1) | | |
| FluA_180817_H3N2_2/3 | SEQ ID NO: 23 | TGAAAATTTGCAGACCTATCAGAA |
| (Influenza A virus, strain | | |
| H3N2) | | |
| H inf primers (Haemophilus | SEQ ID NO: 24 | CGCCAATACATTCAACAAGA |
| influenzae) | | |
| HAV primers (Hepatitis A | SEQ ID NO: 25 | GCATGGAGCTGTAGGAGTCT |
| virus) | | |
| HBV primers (Hepatitis B | SEQ ID NO: 26 | TCCTCACAATACCGCAGAGT |
| virus) | | |
| HCV O Pr1 (Hepatitis C virus) | SEQ ID NO: 27 | ATGAGTGTCGTGCAGCCT |
| HCV Pr4 (Hepatitis C virus) | SEQ ID NO: 28 | CGCAGAAAGCGTCTAGCC |
| HCV Pr5 (Hepatitis C virus) | SEQ ID NO: 29 | GTATGAGTGTCGTGCAGCC |
| HCV Pr6 (Hepatitis C virus) | SEQ ID NO: 30 | CGGGAGAGCCATAGTGGTC |
| HCV 10 Primers (Hepatitis C | SEQ ID NO: 31 | GTATGAGTGTCGTGCAGCC |
| virus) | | |
| HIV primer 1 (Human | SEQ ID NO: 32 | GGTTTATTACAGGGACAGCA |
| immunodeficiency virus-1) | | |
| Malaria primers (All species | SEQ ID NO: 33 | CATGTCGTCTCATCGCAG |
| of plasmodium) | | |
| MS2 primers (Bacteriophage | SEQ ID NO: 34 | TGTCATGGGATCCGGATGTT |
| MS2) | | |
| Parvo primers (Parvovirus | SEQ ID NO: 35 | TGGACAGTTATCTGACCAC |
| B19) | | |
| RSV A_B 4 primers | SEQ ID NO: 36 | TTATGTTAGGACACGCTAGT |
| (Respiratory syncytial virus) | | |
| RSV Pr1 (Respiratory | SEQ ID NO: 37 | TGTTTATGAATGCCTATGGTG |
| syncytial virus) | | |
| Sal_2 primers (Salmonella | SEQ ID NO: 38 | GCGAAGCGTACTGGAAAGG |
| typhimurium, strain LT2) | | |
| TB_3 primers (Mycobacterium | SEQ ID NO: 39 | TCACCTATGTGTCGACCTGG |
| tuberculosis) | | |
| Zika Pr1 (Zika virus) | SEQ ID NO: 40 | AGAGCAGGCCTTGCTACT |
| Zika_2 (Zika virus) | SEQ ID NO: 41 | GCGACCTGATGGTTCTCATC |
| Zika Pr3 (Zika virus) | SEQ ID NO: 42 | GCGACCTGATGGTTCTCATC |

TABLE 15
B3 primers

| Primer set (pathogen) | SEQ ID NO | Sequence |
|---|---|---|
| Dengue Pr1 (Dengue virus) | SEQ ID NO: 43 | CTTCTTGAATGAGCCCCAT |
| Dengue Pr2 (Dengue virus) | SEQ ID NO: 44 | TTCTTGAATGAGCCCCAT |
| Dev2 primers (artificial) | SEQ ID NO: 45 | CGGAAGTTTTCCGCCAAT |
| FluAH3N1_5 primers (Influenza | SEQ ID NO: 46 | CCATTCCCATTNAGGGCATT |
| A virus, strain H3N1) | | |
| FluA_180817_H3N2_2/3 | SEQ ID NO: 47 | ACTCTATGCTGACAAAATGATT |
| (Influenza A virus, strain H3N2) | | |
| H inf primers (Haemophilus | SEQ ID NO: 48 | CGTATGGGGTTTGTGCA |
| influenzae) | | |
| HAV primers (Hepatitis A virus) | SEQ ID NO: 49 | TCCACTGGATGAGAGTCAGT |
| HBV primers (Hepatitis B virus) | SEQ ID NO: 50 | GCATAGCAGCAGGATGAAGA |
| HCV O Pr1 (Hepatitis C virus) | SEQ ID NO: 51 | CACGGTCTACGAGACCTCC |
| HCV Pr4 (Hepatitis C virus) | SEQ ID NO: 52 | CAGGCAGTACCACAAGGC |
| HCV Pr5 (Hepatitis C virus) | SEQ ID NO: 53 | ACCCTATCAGGCAGTACCAC |
| HCV Pr6 (Hepatitis C virus) | SEQ ID NO: 54 | CACGGTCTACGAGACCTCC |
| HCV 10 Primers (Hepatitis C | SEQ ID NO: 55 | CACGGTCTACGAGACCTCC |
| virus) | | |
| HIV primer 1 (Human | SEQ ID NO: 56 | ATCCTGTCTACTTGCCACA |
| immunodeficiency virus-1 | | |
| Malaria primers (All species | SEQ ID NO: 57 | TGGTAATAGGGCTTAAACCAA |
| of plasmodium) | | |
| MS2 primers (Bacteriophage | SEQ ID NO: 58 | CAATAGAGCCGCTCTCAGAG |
| MS2) | | |
| Parvo primers (Parvovirus B19) | SEQ ID NO: 59 | CATGAATCCTTGCAGCAC |
| RSV A_B 4 primers | SEQ ID NO: 60 | TCTTGATTCCTTGGTGTACC |
| (Respiratory syncytial virus) | | |
| RSV Pr1 (Respiratory syncytial | SEQ ID NO: 61 | GTGAGGAAATTGAGTCAAAGA |
| virus) | | |
| Sal_2 primers (Salmonella | SEQ ID NO: 62 | TCAACAATGCGGGGATCTG |
| typhimurium, strain LT2) | | |
| TB_3 primers (Mycobacterium | SEQ ID NO: 63 | CCGGATCGATGTGTACTGAG |
| tuberculosis) | | |
| Zika Pr1 (Zika virus) | SEQ ID NO: 64 | AGCCAATGCGCATATCAGG |
| Zika_2 (Zika virus) | SEQ ID NO: 65 | CAGGGCCATGACAAATGGT |
| Zika Pr3 (Zika virus) | SEQ ID NO: 66 | CAGGGCCATGACAAATGGT |

TABLE 16
FIP primers

| Primer set (pathogen) | SEQ ID NO | Sequence |
|---|---|---|
| Dengue Pr1 (Dengue | SEQ ID NO: 67 | CTCTTCGCCAACTGTGAAACAGTCGAC |
| virus) | | CGTCTTTCAATATGC |
| Dengue Pr2 (Dengue | SEQ ID NO: 68 | CTCTTCGCCAACTGTGAAACAAAACGC |
| virus) | | GCGAGAAACCG |
| Dev2 primers (artificial) | SEQ ID NO: 69 | ACGTAGATCCGTTCCGTTGAAGTTGAC |
| | | CTGGAGATCAAGGA |
| FluAH3N1_5 primers | SEQ ID NO: 70 | TAGCCATTCCATGAGNGCCTCACCCCTC |
| (Influenza A virus, strain | | AAAGCCGAGAT |
| H3N1) | | |
| FluA_180817_H3N2_2/3 | SEQ ID NO: 71 | AAGTGCAAGATCCCAATGATATTGCAG |
| (Influenza A virus, | | ATGCAACGATTCAAGT |
| strain H3N2) | | |
| H inf primers | SEQ ID NO: 72 | ACTTCTTTACCAAAGGCATCATTTTGCG |
| (Haemophilus influenzae) | | TTTGTTGACGCCAAATTCTGG |
| HAV primers (Hepatitis | SEQ ID NO: 73 | CGTAGCCTACCCCTTGTGGAAGGTTTG |
| A virus) | | GAACGTCACCTTGCA |
| HBV primers (Hepatitis | SEQ ID NO: 74 | GTTGGGGACTGCGAATTTTGGCTCGTG |
| B virus) | | GTGGACTTCTCTCAA |
| HCV O Pr1 (Hepatitis C | SEQ ID NO: 75 | ATCCAAGAAAGGACCCGGTCGTCGGGA |
| virus) | | GAGCCATAGTGGT |
| HCV Pr4 (Hepatitis C | SEQ ID NO: 76 | GGTTCCGCAGACCACTATGGCATGAGT |
| virus) | | GTCGTGCAGCCT |
| HCV Pr5 (Hepatitis C | SEQ ID NO: 77 | CAAGAAAGGACCCGGTCGTCCCGGGAG |
| virus) | | AGCCATAGTGGT |
| HCV Pr6 (Hepatitis C | SEQ ID NO: 78 | GCCCAAATCTCCAGGCATTGAGCGGAA |
| virus) | | CCGGTGAGTACAC |
| HCV 10 Primers | SEQ ID NO: 79 | CAAGAAAGGACCCGGTCGTCCCGGGAG |
| (Hepatitis C virus) | | AGCCATAGTGGT |
| HIV primer 1 (Human | SEQ ID NO: 80 | TCTTGTATTACTACTGCCCCTTCAAATC |
| immunodeficiency virus- | | CACTTTGGAAAGGACC |
| 1) | | |
| Malaria primers (All | SEQ ID NO: 81 | GCCTGGAGTTCTATACCCAGTATAGCG |
| species of plasmodium) | | TGTATTGTTGCCTT |
| MS2 primers | SEQ ID NO: 82 | GCCCAAACAACGACGATCGGTAAAACC |
| (Bacteriophage MS2) | | AGCATCCGTAGCCT |
| Parvo primers | SEQ ID NO: 83 | AGGCTTGTGTAAGTCTTCACTAGATCCC |
| (Parvovirus B19) | | CATGCCTTATCATCC |
| RSV A_B 4 primers | SEQ ID NO: 84 | ATATGGTAGAATCCTGCTTCTCCACTAC |
| (Respiratory syncytial | | AAGCAGAAATGGAACAAGT |
| virus) | | |
| RSV Pr1 (Respiratory | SEQ ID NO: 85 | GCACACTAGCATGTCCTAACATAATCA |
| syncytial virus) | | GGGCAAGTGATGTTACG |
| Sal_2 primers | SEQ ID NO: 86 | ATGATGCCGGCAATAGCGTCACAAAGC |
| (Salmonella | | CAGCTTTACGGTTCC |
| typhimurium, strain LT2) | | |
| TB_3 primers | SEQ ID NO: 87 | TGGAGGTGGCCATCGTGGAACCTACGT |
| (Mycobacterium | | GGCCTTTGTCAC |
| tuberculosis) | | |
| Zika Pr1 (Zika virus) | SEQ ID NO: 88 | GTTAGTCCCAGGGCCATGACAAGGGGG |
| | | GTTTATGCTCCTCT |
| Zika _2 (Zika virus) | SEQ ID NO: 89 | AGCCAGGATTGCCAAGGTGATGGTTGG |
| | | CAATACGAGCGAT |
| Zika Pr3 (Zika virus) | SEQ ID NO: 90 | GCCAGGATTGCCAAGGTGATGTTTTGC |
| | | TTTGGCCTGGTTGG |

TABLE 17
BIP primers

| Primer set (pathogen) | SEQ ID NO | Sequence |
|---|---|---|
| Dengue Pr1 (Dengue | SEQ ID NO: 91 | GGACCCATGAAATTGGTGATGGAGCCA |
| virus) | | AAATTCCTGCTGT |
| Dengue Pr2 (Dengue | SEQ ID NO: 92 | GGACCCATGAAATTGGTGATGGAGCCA |
| virus) | | AAATTCCTGCTGT |
| Dev2 primers (artificial) | SEQ ID NO: 93 | CAGCCTGCATAATGAAAACGGAGACAA |
| | | CAGACAGAACCCAA |
| FluAH3N1_5 primers | SEQ ID NO: 94 | AGACCAATCYTGTCACCTCTGACGTCT |
| (Influenza A virus, strain | | ACGCTGCAGTCCT |
| H3N1) | | |
| FluA_180817_H3N2_2/3 | SEQ ID NO: 95 | GAAGGAGTACCTGAGTCTATGTCGTCA |
| (Influenza A virus, | | GCATCCACAGC |
| strain H3N2) | | |
| H inf primers | SEQ ID NO: 96 | CTGATGATATGGGTACATCTGTTCGCG |
| (Haemophilus influenzae) | | AAGAATGAGAAGTTTTGTGG |
| HAV primers (Hepatitis | SEQ ID NO: 97 | TGCCTTGGATAGGGTAACAGCGCTCCG |
| A virus) | | GCGTTGAATGGTT |
| HBV primers (Hepatitis | SEQ ID NO: 98 | TCACCAACCTCCTGTCCTCCAAATAAA |
| B virus) | | ACGCCGCAGACACAT |
| HCV O Pr1 (Hepatitis C | SEQ ID NO: 99 | CGCGAGACTGCTAGCCGAGTAGCAAGC |
| virus) | | ACCCTATCAGGC |
| HCV Pr4 (Hepatitis C | SEQ ID NO: 100 | TTGGATCAACCCGCTCAATGCCACCCA |
| virus) | | ACACTACTCGGCT |
| HCV Pr5 (Hepatitis C | SEQ ID NO: 101 | AACCCGCTCAATGCCTGGAGGCGACCC |
| virus) | | AACACTACTCG |
| HCV Pr6 (Hepatitis C | SEQ ID NO: 102 | TAGCCGAGTAGTGTTGGGTCGCACTCG |
| virus) | | CAAGCACCCTAT |
| HCV 10 Primers | SEQ ID NO: 103 | CGCGAGACTGCTAGCCGAGTAGCAAGC |
| (Hepatitis C virus) | | ACCCTATCAGGC |
| HIV primer 1 (Human | SEQ ID NO: 104 | AGTGACATAAAAGTAGTGCCAAGAAAT |
| immunodeficiency virus- | | CATCACCTGCCATCTG |
| 1) | | |
| Malaria primers (All | SEQ ID NO: 105 | ACAGCCGGAAAGGTAATTTTACGAACA |
| species of plasmodium) | | TTTTTTAGTCCCATGCTA |
| MS2 primers | SEQ ID NO: 106 | GCACGTTCTCCAACGGTGCTGGTTGCTT |
| (Bacteriophage MS2) | | GTTCAGCGAACT |
| Parvo primers | SEQ ID NO: 107 | GTTAGCGTACAACTACCCGGTATGTCA |
| (Parvovirus B19) | | ACAGCACTTTGCG |
| RSV A_B 4 primers | SEQ ID NO: 108 | TCAATTTCCTCACTTCTCTAGTGTTCTG |
| (Respiratory syncytial | | TATTCTCCCATTATGCC |
| virus) | | |
| RSV Pr1 (Respiratory | SEQ ID NO: 109 | TGGAACAAGTTGTTGAGGTTTATGAGG |
| syncytial virus) | | TTGTTCAATATATGGTAGAATCC |
| Sal_2 primers | SEQ ID NO: 110 | GTGGGGATGACTCGCCATGGACCATCA |
| (Salmonella | | CCAATGGTCAGC |
| typhimurium, strain LT2) | | |
| TB_3 primers | SEQ ID NO: 111 | CCATCTGGACCCGCCAACAACCCCTAT |
| (Mycobacterium | | CCGTATGGTGGA |
| tuberculosis) | | |
| Zika Pr1 (Zika virus) | SEQ ID NO: 112 | AACGTGGTGGGACTGCTGTTACAGCTG |
| | | TGAGTACTTCGCT |
| Zika_2 (Zika virus) | SEQ ID NO: 113 | TGGAGAGCAGGCCTTGCTACTTCACTG |
| | | CCTTTTCCCTTCAGA |
| Zika Pr3 (Zika virus) | SEQ ID NO: 114 | TGGAGAGCAGGCCTTGCTACTTCACTG |
| | | CCTTTTCCCTTCAGA |

TABLE18
LF primers

| Primer set (pathogen) | SEQ ID NO | Sequence |
|---|---|---|
| FluAH3N1_5 primers | SEQ ID NO: 118 | TGCAAATACAYYTTCCAGTCTCTG |
| (Influenza A virus, strain | | |
| H3N1) | | |
| FluA_180817_H3N2_2/3 | SEQ ID NO: 119 | GCGGCAACAACAAGCGGGTC |
| (Influenza A virus, strain | | |
| H3N2) | | |
| H inf primers | SEQ ID NO: 120 | GCAGACGACCAAAGGTATCTTG |
| (Haemophilus influenzae) | | |
| HAV primers (Hepatitis | SEQ ID NO: 121 | CAAAGAGATTCATGAAAGCCAAG |
| A virus) | | |
| HBV primers (Hepatitis | SEQ ID NO: 122 | CACGGGTGATCCCCCTA |
| B virus) | | |
| HCV O Pr1 (Hepatitis C | SEQ ID NO: 123 | TGTACTCACCGGTTCCGCAG |
| virus) | | |
| HCV Pr4 (Hepatitis C | SEQ ID NO: 124 | none |
| virus) | | |
| HCV Pr5 (Hepatitis C | SEQ ID NO: 125 | GTGTACTCACCGGTTCCGCAG |
| virus) | | |
| HCV Pr6 (Hepatitis C | SEQ ID NO: 126 | GGTCGTCCTGGCAATTCCG |
| virus) | | |
| HCV 10 Primers | SEQ ID NO: 127 | GTGTACTCACCGGTTCCGCAG |
| (Hepatitis C virus) | | |
| HIV primer 1 (Human | SEQ ID NO: 128 | CTTTCCAGAGGAGCTTTGCT |
| immunodeficiency virus- | | |
| 1) | | |
| Malaria primers (All | SEQ ID NO: 129 | CGTGACGAGCGGTGTGTAC |
| species of plasmodium) | | |
| MS2 primers | SEQ ID NO: 130 | CCAGAGAGGAGGTTGCCAA |
| (Bacteriophage MS2) | | |
| Parvo primers | SEQ ID NO: 131 | TTCTCCTCTAGGTTCTGCATGA |
| (Parvovirus B19) | | |
| RSV A_B 4 primers | SEQ ID NO: 132 | GAGCATACTCATACACCTCCACA |
| (Respiratory syncytial | | |
| virus) | | |
| RSV pr1 (Respiratory | SEQ ID NO: 133 | CTGATTTTGCTAAGACTCCCCAC |
| syncytial virus) | | |
| Sal_2 primers | | |
| (Salmonella | SEQ ID NO: 134 | TGATAAACTTCATCGCACCGTC |
| typhimurium, strain LT2) | | |
| TB_3 primers | | |
| (Mycobacterium | SEQ ID NO: 135 | AGGATCCTGCGACGTAGGCG |
| tuberculosis) | | |
| Zika Pr1 (Zika virus) | SEQ ID NO: 136 | AAGTTCTTCTTCACACTGCCTTTTC |
| Zika_2 (Zika virus) | SEQ ID NO: 137 | TTATCAGTGCGTGGAACAACC |
| Zika Pr3 (Zika virus) | SEQ ID NO: 138 | TCAGTGCGTGGAACAACC |

TABLE 19
LB primers

| Primer set (pathogen) | SEQ ID NO | Sequence |
|---|---|---|
| Dengue Pr1 (Dengue | SEQ ID NO: 139 | CCTAAGATTTCTAGCCATACCTCCA |
| virus) | | |
| Dengue Pr2 (Dengue | SEQ ID NO: 140 | CCTAAGATTTCTAGCCATACCTCCA |
| virus) | | |
| Dev2 primers (artificial) | SEQ ID NO: 141 | TTGCCGACGACGAAAGCGA |
| FluAH3N1_5 primers | SEQ ID NO: 142 | GGATTTGTGTTCACGCTCACCG |
| (Influenza A virus, strain | | |
| H3N1) | | |
| FluA_180817_H3N2_2/3 | SEQ ID NO: 143 | AGGGAAGAATATCGAAAGGAA |
| (Influenza A virus, strain | | |
| H3N2) | | |
| H inf primers | SEQ ID NO: 144 | none |
| (Haemophilus influenzae) | | |
| HAV primers (Hepatitis | SEQ ID NO: 145 | CGGATATTGGTGAGTTGTTAAGACA |
| A virus) | | |
| HBV primers (Hepatitis | SEQ ID NO: 146 | TTTGTCCTGGTTATCGCTGG |
| B virus) | | |
| HCV O Pr1 (Hepatitis C | SEQ ID NO: 147 | TTGGGTCGCGAAAGGCC |
| virus) | | |
| HCV Pr4 (Hepatitis C | SEQ ID NO: 148 | TGGAGATTTGGGCGTGC |
| virus) | | |
| HCV Pr5 (Hepatitis C | SEQ ID NO: 149 | TGCCCCCGCAAGACTGCTA |
| virus) | | |
| HCV Pr6 (Hepatitis C | SEQ ID NO: 150 | CGAAAGGCCTTGTGGTACTG |
| virus) | | |
| HCV 10 Primers | SEQ ID NO: 151 | TTGGGTCGCGAAAGGCC |
| (Hepatitis C virus) | | |
| HIV primer 1 (Human | SEQ ID NO: 152 | GCAAAGATCATTAGGGATTATGGAA |
| immunodeficiency virus- | | |
| 1) | | |
| Malaria primers (All | SEQ ID NO: 153 | CCCTTAACGTAAAGATCATTTATGAA |
| species of plasmodium) | | |
| MS2 primers | SEQ ID NO: 154 | TGCAGGATGCAGCGCCTTA |
| (Bacteriophage MS2) | | |
| Parvo primers | SEQ ID NO: 155 | TAACTATGTTGGGCCTGGCA |
| (Parvovirus B19) | | |
| RSV A_B 4 primers | SEQ ID NO: 156 | TATTGGGCAATGCTGCTGGC |
| (Respiratory syncytial | | |
| virus) | | |
| RSV Pr1 (Respiratory | SEQ ID NO: 157 | CCAAAAATTGGGTGGTGAAGCA |
| syncytial virus) | | |
| Sal_2 primers | | |
| (Salmonella | SEQ ID NO: 158 | TATGGATTTGTCCTCCGCCCT |
| typhimurium, strain LT2) | | |
| TB_3 primers | | |
| (Mycobacterium | SEQ ID NO: 159 | GAAGGCGTACTCGACCTGAAAGAC |
| tuberculosis) | | |
| Zika Pr1 (Zika virus) | SEQ ID NO: 160 | TCACAAGGAGTGGGAAGCG |
| Zika_2 (Zika virus) | SEQ ID NO: 161 | GGGGGGTTTATGCTCCTCTC |
| Zika Pr3 (Zika virus) | SEQ ID NO: 162 | GCGGGGGGTTTATGCTC |

The term “comprising” as used herein is synonymous with “including,” “containing,” or “characterized by,” and is inclusive or open-ended and does not exclude additional, unrecited elements or method steps.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1. An aqueous solution comprising:
a set of loop-mediated isothermal amplification (LAMP) primers sufficient to perform a LAMP reaction of a target nucleic acid;
a polymerase; and
a first inhibitor oligonucleotide comprising a hairpin, wherein:
the first inhibitor oligonucleotide does not specifically hybridize to the target nucleic acid, and
the first inhibitor oligonucleotide has activity to reduce the level of a nonspecific amplification product of the LAMP reaction compared to the level of a nonspecific amplification product of a LAMP reaction performed in the absence of the first inhibitor oligonucleotide.

2-85. (canceled)


SEQUENCE LISTING (162 sequences)

SEQ ID NO: 1 - DNA, 42 bp, Artificial Sequence
  HAVFIP1
cgtagcctaccccttgtggaaggtttggaacgtcaccttgca

SEQ ID NO: 2 - DNA, 46 bp, Artificial Sequence
  # HAVFIP1_4b ext_Phos3′ (aka Phospho-FIP4)
cgtagcctaccccttgtggaaggtttggaacgtcaccttgcacaag

SEQ ID NO: 3 - DNA, 43 bp, Artificial Sequence
  # HAVFIP1_1b ext Phos3′
cgtagcctaccccttgtggaaggtttggaacgtcaccttgcac

SEQ ID NO: 4 - DNA, 44 bp, Artificial Sequence
  # HAVFIP1_2b ext_Phos3′
cgtagcctaccccttgtggaaggtttggaacgtcaccttgcaca

SEQ ID NO: 5 - DNA, 45 bp, Artificial Sequence
  # HAVFIP1_3b ext_Phos3′
cgtagcctaccccttgtggaaggtttggaacgtcaccttgcacaa

SEQ ID NO: 6 - DNA, 50 bp, Artificial Sequence
  # HAVFIP1_8b ext_Phos3′ (aka Phospho-FIP8)
cgtagcctaccccttgtggaaggtttggaacgtcaccttgcacaaggggt

SEQ ID NO: 7 - DNA, 54 bp, Artificial Sequence
  # HAVFIP1_12b ext_Phos3′ (aka Phospho-FIP12)
cgtagcctaccccttgtggaaggtttggaacgtcaccttgcacaaggggtaggc

SEQ ID NO: 8 - DNA, 58 bp, Artificial Sequence
  # HAVFIP1_16b full ext_Phos3′ (aka Phospho-FIP16)
cgtagcctaccccttgtggaaggtttggaacgtcaccttgcacaaggggtaggctacg

SEQ ID NO: 9 - DNA, 26 bp, Artificial Sequence
  HAVFIP1_hairpin
tggaaggtttggaacgtcaccttgca

SEQ ID NO: 10 - DNA, 42 bp, Artificial Sequence
  HAVFIP1_early complement
gcatcggatggggaactggaaggtttggaacgtcaccttgca

SEQ ID NO: 11 - DNA, 40 bp, Artificial Sequence
  Dev3 BIP_4b ext
ggcaagtggcagcccaaacggagcccccagcatcgtttgg

SEQ ID NO: 12 - DNA, 53 bp, Artificial Sequence
  Dev3 BIP_17b full ext
ggcaagtggcagcccaaacggagcccccagcatcgtttgggctgccacttgcc

SEQ ID NO: 13 - DNA, 38 bp, Artificial Sequence
  HAVFIP1_4b cut
cgtagcctaccccttgtggaaggtttggaacgtcacct

SEQ ID NO: 14 - DNA, 50 bp, Artificial Sequence
  RSV Pr2 FIP
taatgatgcttttgggttgttcaatgtttatgaatatgcccaaaaattgg

SEQ ID NO: 15 - DNA, 36 bp, Artificial Sequence
  Dev3 BIP
ggcaagtggcagcccaaacggagcccccagcatcgt

SEQ ID NO: 16 - DNA, 41 bp, Artificial Sequence
  TB Pr2 BIP
tctaccagtactgcggcgacgttctctggcgttgagcgtag

SEQ ID NO: 17 - DNA, 40 bp, Artificial Sequence
  HAVFIP1_2b cut
cgtagcctaccccttgtggaaggtttggaacgtcaccttg

SEQ ID NO: 18 - DNA, 26 bp, Artificial Sequence
  HAVFIP1_hairpin mirror
accttccaaaccttgcagtggaacgt

SEQ ID NO: 19 - DNA, 20 bp, Artificial Sequence
  Dengue Pr1 (Dengue virus)
tctctgatgaacaaccaacg

SEQ ID NO: 20 - DNA, 20 bp, Artificial Sequence
  Dengue Pr2 (Dengue virus)
gaccgtctttcaatatgctg

SEQ ID NO: 21 - DNA, 18 bp, Artificial Sequence
  Dev2 primers (artificial)
cgtgtttgctctcacgaa

SEQ ID NO: 22 - DNA, 17 bp, Artificial Sequence
  FluAH3N1_5 primers (Influenza A virus, strain H3N1)
ctatcrtcccgtcaggc

SEQ ID NO: 23 - DNA, 24 bp, Artificial Sequence
  FluA_180817_H3N2_2/3 (Influenza A virus, strain H3N2)
tgaaaatttgcagacctatcagaa

SEQ ID NO: 24 - DNA, 20 bp, Artificial Sequence
  H inf primers (Haemophilus influenzae)
cgccaatacattcaacaaga

SEQ ID NO: 25 - DNA, 20 bp, Artificial Sequence
  HAV primers (Hepatitis A virus)
gcatggagctgtaggagtct

SEQ ID NO: 26 - DNA, 20 bp, Artificial Sequence
  HBV primers (Hepatitis B virus)
tcctcacaataccgcagagt

SEQ ID NO: 27 - DNA, 18 bp, Artificial Sequence
  HCV O Pr1 (Hepatitis C virus)
atgagtgtcgtgcagcct

SEQ ID NO: 28 - DNA, 18 bp, Artificial Sequence
  HCV Pr4 (Hepatitis C virus)
cgcagaaagcgtctagcc

SEQ ID NO: 29 - DNA, 19 bp, Artificial Sequence
  HCV Pr5 (Hepatitis C virus)
gtatgagtgtcgtgcagcc

SEQ ID NO: 30 - DNA, 19 bp, Artificial Sequence
  HCV Pr6 (Hepatitis C virus)
cgggagagccatagtggtc

SEQ ID NO: 31 - DNA, 19 bp, Artificial Sequence
  HCV 10 Primers (Hepatitis C virus)
gtatgagtgtcgtgcagcc

SEQ ID NO: 32 - DNA, 20 bp, Artificial Sequence
  HIV primer 1 (Human immunodeficiency virus-1)
ggtttattacagggacagca

SEQ ID NO: 33 - DNA, 18 bp, Artificial Sequence
  Malaria primers (All species of plasmodium)
catgtcgtctcatcgcag

SEQ ID NO: 34 - DNA, 20 bp, Artificial Sequence
  MS2 primers (Bacteriophage MS2)
tgtcatgggatccggatgtt

SEQ ID NO: 35 - DNA, 19 bp, Artificial Sequence
  Parvo primers (Parvovirus B19)
tggacagttatctgaccac

SEQ ID NO: 36 - DNA, 20 bp, Artificial Sequence
  RSV A_B 4 primers (Respiratory syncytial virus)
ttatgttaggacacgctagt

SEQ ID NO: 37 - DNA, 21 bp, Artificial Sequence
  RSV Pr1 (Respiratory syncytial virus)
tgtttatgaatgcctatggtg

SEQ ID NO: 38 - DNA, 19 bp, Artificial Sequence
  Sal_2 primers (Salmonella typhimurium, strain LT2)
gcgaagcgtactggaaagg

SEQ ID NO: 39 - DNA, 20 bp, Artificial Sequence
  TB_3 primers (Mycobacterium tuberculosis)
tcacctatgtgtcgacctgg

SEQ ID NO: 40 - DNA, 18 bp, Artificial Sequence
  Zika Pr1 (Zika virus)
agagcaggccttgctact

SEQ ID NO: 41 - DNA, 20 bp, Artificial Sequence
  Zika_2 (Zika virus)
gcgacctgatggttctcatc

SEQ ID NO: 42 - DNA, 20 bp, Artificial Sequence
  Zika Pr3 (Zika virus)
gcgacctgatggttctcatc

SEQ ID NO: 43 - DNA, 19 bp, Artificial Sequence
  Dengue Pr1 (Dengue virus)
cttcttgaatgagccccat

SEQ ID NO: 44 - DNA, 18 bp, Artificial Sequence
  Dengue Pr2 (Dengue virus)
ttcttgaatgagccccat

SEQ ID NO: 45 - DNA, 18 bp, Artificial Sequence
  Dev2 primers (artificial)
cggaagttttccgccaat

SEQ ID NO: 46 - DNA, 20 bp, Artificial Sequence
  FluAH3N1_5 primers (Influenza A virus, strain H3N1)
  misc_feature (0)...(0): N is any nucleotide
ccattcccattnagggcatt

SEQ ID NO: 47 - DNA, 22 bp, Artificial Sequence
  FluA_180817_H3N2_2/3 (Influenza A virus, strain H3N2)
actctatgctgacaaaatgatt

SEQ ID NO: 48 - DNA, 17 bp, Artificial Sequence
  H inf primers (Haemophilus influenzae)
cgtatggggtttgtgca

SEQ ID NO: 49 - DNA, 20 bp, Artificial Sequence
  HAV primers (Hepatitis A virus)
tccactggatgagagtcagt

SEQ ID NO: 50 - DNA, 20 bp, Artificial Sequence
  HBV primers (Hepatitis B virus)
gcatagcagcaggatgaaga

SEQ ID NO: 51 - DNA, 19 bp, Artificial Sequence
  HCV O Pr1 (Hepatitis C virus)
cacggtctacgagacctcc

SEQ ID NO: 52 - DNA, 18 bp, Artificial Sequence
  HCV Pr4 (Hepatitis C virus)
caggcagtaccacaaggc

SEQ ID NO: 53 - DNA, 20 bp, Artificial Sequence
  HCV Pr5 (Hepatitis C virus)
accctatcaggcagtaccac

SEQ ID NO: 54 - DNA, 19 bp, Artificial Sequence
  HCV Pr6 (Hepatitis C virus)
cacggtctacgagacctcc

SEQ ID NO: 55 - DNA, 19 bp, Artificial Sequence
  HCV 10 Primers (Hepatitis C virus)
cacggtctacgagacctcc

SEQ ID NO: 56 - DNA, 19 bp, Artificial Sequence
  HIV primer 1 (Human immunodeficiency virus-1)
atcctgtctacttgccaca

SEQ ID NO: 57 - DNA, 21 bp, Artificial Sequence
  Malaria primers (All species of plasmodium)
tggtaatagggcttaaaccaa

SEQ ID NO: 58 - DNA, 20 bp, Artificial Sequence
  MS2 primers (Bacteriophage MS2)
caatagagccgctctcagag

SEQ ID NO: 59 - DNA, 18 bp, Artificial Sequence
  Parvo primers (Parvovirus B19)
catgaatccttgcagcac

SEQ ID NO: 60 - DNA, 20 bp, Artificial Sequence
  RSV A_B 4 primers (Respiratory syncytial virus)
tcttgattccttggtgtacc

SEQ ID NO: 61 - DNA, 21 bp, Artificial Sequence
  RSV Pr1 (Respiratory syncytial virus)
gtgaggaaattgagtcaaaga

SEQ ID NO: 62 - DNA, 19 bp, Artificial Sequence
  Sal_2 primers (Salmonella typhimurium, strain LT2)
tcaacaatgcggggatctg

SEQ ID NO: 63 - DNA, 20 bp, Artificial Sequence
  TB_3 primers (Mycobacterium tuberculosis)
ccggatcgatgtgtactgag

SEQ ID NO: 64 - DNA, 19 bp, Artificial Sequence
  Zika Pr1 (Zika virus)
agccaatgcgcatatcagg

SEQ ID NO: 65 - DNA, 19 bp, Artificial Sequence
  Zika_2 (Zika virus)
cagggccatgacaaatggt

SEQ ID NO: 66 - DNA, 19 bp, Artificial Sequence
  Zika Pr3 (Zika virus)
cagggccatgacaaatggt

SEQ ID NO: 67 - DNA, 42 bp, Artificial Sequence
  Dengue Pr1 (Dengue virus)
ctcttcgccaactgtgaaacagtcgaccgtctttcaatatgc

SEQ ID NO: 68 - DNA, 38 bp, Artificial Sequence
  Dengue Pr2 (Dengue virus)
ctcttcgccaactgtgaaacaaaacgcgcgagaaaccg

SEQ ID NO: 69 - DNA, 41 bp, Artificial Sequence
  Dev2 primers (artificial)
acgtagatccgttccgttgaagttgacctggagatcaagga

SEQ ID NO: 70 - DNA, 39 bp, Artificial Sequence
  FluAH3N1_5 primers (Influenza A virus, strain H3N1)
  misc_feature (0)...(0): N is any nucleotide
tagccattccatgagngcctcacccctcaaagccgagat

SEQ ID NO: 71 - DNA, 43 bp, Artificial Sequence
  FluA_180817_H3N2_2/3 (Influenza A virus, strain H3N2)
aagtgcaagatcccaatgatattgcagatgcaacgattcaagt

SEQ ID NO: 72 - DNA, 49 bp, Artificial Sequence
  H inf primers (Haemophilus influenzae)
acttctttaccaaaggcatcattttgcgtttgttgacgccaaattctgg

SEQ ID NO: 73 - DNA, 42 bp, Artificial Sequence; identical to SEQ ID NO: 1
  HAV primers (Hepatitis A virus)

SEQ ID NO: 74 - DNA, 42 bp, Artificial Sequence
  HBV primers (Hepatitis B virus)
gttggggactgcgaattttggctcgtggtggacttctctcaa

SEQ ID NO: 75 - DNA, 40 bp, Artificial Sequence
  HCV O Pr1 (Hepatitis C virus)
atccaagaaaggacccggtcgtcgggagagccatagtggt

SEQ ID NO: 76 - DNA, 39 bp, Artificial Sequence
  HCV Pr4 (Hepatitis C virus)
ggttccgcagaccactatggcatgagtgtcgtgcagcct

SEQ ID NO: 77 - DNA, 39 bp, Artificial Sequence
  HCV Pr5 (Hepatitis C virus)
caagaaaggacccggtcgtcccgggagagccatagtggt

SEQ ID NO: 78 - DNA, 40 bp, Artificial Sequence
  HCV Pr6 (Hepatitis C virus)
gcccaaatctccaggcattgagcggaaccggtgagtacac

SEQ ID NO: 79 - DNA, 39 bp, Artificial Sequence; identical to SEQ ID NO: 77
  HCV 10 Primers (Hepatitis C virus)

SEQ ID NO: 80 - DNA, 44 bp, Artificial Sequence
  HIV primer 1 (Human immunodeficiency virus-1)
tcttgtattactactgccccttcaaatccactttggaaaggacc

SEQ ID NO: 81 - DNA, 41 bp, Artificial Sequence
  Malaria primers (All species of plasmodium)
gcctggagttctatacccagtatagcgtgtattgttgcctt

SEQ ID NO: 82 - DNA, 41 bp, Artificial Sequence
  MS2 primers (Bacteriophage MS2)
gcccaaacaacgacgatcggtaaaaccagcatccgtagcct

SEQ ID NO: 83 - DNA, 43 bp, Artificial Sequence
  Parvo primers (Parvovirus B19)
aggcttgtgtaagtcttcactagatccccatgccttatcatcc

SEQ ID NO: 84 - DNA, 47 bp, Artificial Sequence
  RSV A_B 4 primers (Respiratory syncytial virus)
atatggtagaatcctgcttctccactacaagcagaaatggaacaagt

SEQ ID NO: 85 - DNA, 44 bp, Artificial Sequence
  RSV Pr1 (Respiratory syncytial virus)
gcacactagcatgtcctaacataatcagggcaagtgatgttacg

SEQ ID NO: 86 - DNA, 42 bp, Artificial Sequence
  Sal_2 primers (Salmonella typhimurium, strain LT2)
atgatgccggcaatagcgtcacaaagccagctttacggttcc

SEQ ID NO: 87 - DNA, 39 bp, Artificial Sequence
  TB_3 primers (Mycobacterium tuberculosis)
tggaggtggccatcgtggaacctacgtggcctttgtcac

SEQ ID NO: 88 - DNA, 41 bp, Artificial Sequence
  Zika Pr1 (Zika virus)
gttagtcccagggccatgacaaggggggtttatgctcctct

SEQ ID NO: 89 - DNA, 40 bp, Artificial Sequence
  Zika_2 (Zika virus)
agccaggattgccaaggtgatggttggcaatacgagcgat

SEQ ID NO: 90 - DNA, 41 bp, Artificial Sequence
  Zika Pr3 (Zika virus)
gccaggattgccaaggtgatgttttgctttggcctggttgg

SEQ ID NO: 91 - DNA, 40 bp, Artificial Sequence
  Dengue Pr1 (Dengue virus)
ggacccatgaaattggtgatggagccaaaattcctgctgt

SEQ ID NO: 92 - DNA, 40 bp, Artificial Sequence; identical to SEQ ID NO: 91
  Dengue Pr2 (Dengue virus)

SEQ ID NO: 93 - DNA, 41 bp, Artificial Sequence
  Dev2 primers (artificial)
cagcctgcataatgaaaacggagacaacagacagaacccaa

SEQ ID NO: 94 - DNA, 40 bp, Artificial Sequence
  FluAH3N1_5 primers (Influenza A virus, strain H3N1)
agaccaatcytgtcacctctgacgtctacgctgcagtcct

SEQ ID NO: 95 - DNA, 38 bp, Artificial Sequence
  FluA_180817_H3N2_2/3 (Influenza A virus, strain H3N2)
gaaggagtacctgagtctatgtcgtcagcatccacagc

SEQ ID NO: 96 - DNA, 47 bp, Artificial Sequence
  H inf primers (Haemophilus influenzae)
ctgatgatatgggtacatctgttcgcgaagaatgagaagttttgtgg

SEQ ID NO: 97 - DNA, 40 bp, Artificial Sequence
  HAV primers (Hepatitis A virus)
tgccttggatagggtaacagcgctccggcgttgaatggtt

SEQ ID NO: 98 - DNA, 42 bp, Artificial Sequence
  HBV primers (Hepatitis B virus)
tcaccaacctcctgtcctccaaataaaacgccgcagacacat

SEQ ID NO: 99 - DNA, 39 bp, Artificial Sequence
  HCV O Pr1 (Hepatitis C virus)
cgcgagactgctagccgagtagcaagcaccctatcaggc

SEQ ID NO: 100 - DNA, 40 bp, Artificial Sequence
  HCV Pr4 (Hepatitis C virus)
ttggatcaacccgctcaatgccacccaacactactcggct

SEQ ID NO: 101 - DNA, 38 bp, Artificial Sequence
  HCV Pr5 (Hepatitis C virus)
aacccgctcaatgcctggaggcgacccaacactactcg

SEQ ID NO: 102 - DNA, 39 bp, Artificial Sequence
  HCV Pr6 (Hepatitis C virus)
tagccgagtagtgttgggtcgcactcgcaagcaccctat

SEQ ID NO: 103 - DNA, 39 bp, Artificial Sequence; identical to SEQ ID NO: 99
  HCV 10 Primers (Hepatitis C virus)

SEQ ID NO: 104 - DNA, 43 bp, Artificial Sequence
  HIV primer 1 (Human immunodeficiency virus-1)
agtgacataaaagtagtgccaagaaatcatcacctgccatctg

SEQ ID NO: 105 - DNA, 45 bp, Artificial Sequence
  Malaria primers (All species of plasmodium)
acagccggaaaggtaattttacgaacattttttagtcccatgcta

SEQ ID NO: 106 - DNA, 40 bp, Artificial Sequence
  MS2 primers (Bacteriophage MS2)
gcacgttctccaacggtgctggttgcttgttcagcgaact

SEQ ID NO: 107 - DNA, 40 bp, Artificial Sequence
  Parvo primers (Parvovirus B19)
gttagcgtacaactacccggtatgtcaacagcactttgcg

SEQ ID NO: 108 - DNA, 45 bp, Artificial Sequence
  RSV A_B 4 primers (Respiratory syncytial virus)
tcaatttcctcacttctctagtgttctgtattctcccattatgcc

SEQ ID NO: 109 - DNA, 50 bp, Artificial Sequence
  RSV Pr1 (Respiratory syncytial virus)
tggaacaagttgttgaggtttatgaggttgttcaatatatggtagaatcc

SEQ ID NO: 110 - DNA, 39 bp, Artificial Sequence
  Sal_2 primers (Salmonella typhimurium, strain LT2)
gtggggatgactcgccatggaccatcaccaatggtcagc

SEQ ID NO: 111 - DNA, 39 bp, Artificial Sequence
  TB_3 primers (Mycobacterium tuberculosis)
ccatctggacccgccaacaacccctatccgtatggtgga

SEQ ID NO: 112 - DNA, 40 bp, Artificial Sequence
  Zika Pr1 (Zika virus)
aacgtggtgggactgctgttacagctgtgagtacttcgct

SEQ ID NO: 113 - DNA, 42 bp, Artificial Sequence
  Zika_2 (Zika virus)
tggagagcaggccttgctacttcactgccttttcccttcaga

SEQ ID NO: 114 - DNA, 42 bp, Artificial Sequence; identical to SEQ ID NO: 113
  Zika Pr3 (Zika virus)

SEQ ID NO: 115 - DNA, 18 bp, Artificial Sequence
  Dengue Pr1 (Dengue virus)
gcggtttctcgcgcgttt

SEQ ID NO: 116 - skipped

SEQ ID NO: 117 - DNA, 19 bp, Artificial Sequence
  Dev2 primers (artificial)
cgctgtccagttcgacaag

SEQ ID NO: 118 - DNA, 24 bp, Artificial Sequence
  FluAH3N1_5 primers (Influenza A virus, strain H3N1)
tgcaaatacayyttccagtctctg

SEQ ID NO: 119 - DNA, 20 bp, Artificial Sequence
  FluA_180817_H3N2_2/3 (Influenza A virus, strain H3N2)
gcggcaacaacaagcgggtc

SEQ ID NO: 120 - DNA, 22 bp, Artificial Sequence
  H inf primers (Haemophilus influenzae)
gcagacgaccaaaggtatcttg

SEQ ID NO: 121 - DNA, 23 bp, Artificial Sequence
  HAV primers (Hepatitis A virus)
caaagagattcatgaaagccaag

SEQ ID NO: 122 - DNA, 17 bp, Artificial Sequence
  HBV primers (Hepatitis B virus)
cacgggtgatcccccta

SEQ ID NO: 123 - DNA, 20 bp, Artificial Sequence
  HCV O Pr1 (Hepatitis C virus)
tgtactcaccggttccgcag

SEQ ID NO: 124 - skipped

SEQ ID NO: 125 - DNA, 21 bp, Artificial Sequence
  HCV Pr5 (Hepatitis C virus)
gtgtactcaccggttccgcag

SEQ ID NO: 126 - DNA, 19 bp, Artificial Sequence
  HCV Pr6 (Hepatitis C virus)
ggtcgtcctggcaattccg

SEQ ID NO: 127 - DNA, 21 bp, Artificial Sequence
  HCV 10 Primers (Hepatitis C virus)
gtgtactcaccggttccgcag

SEQ ID NO: 128 - DNA, 20 bp, Artificial Sequence
  HIV primer 1 (Human immunodeficiency virus-1)
ctttccagaggagctttgct

SEQ ID NO: 129 - DNA, 19 bp, Artificial Sequence
  Malaria primers (All species of plasmodium)
cgtgacgagcggtgtgtac

SEQ ID NO: 130 - DNA, 19 bp, Artificial Sequence
  MS2 primers (Bacteriophage MS2)
ccagagaggaggttgccaa

SEQ ID NO: 131 - DNA, 22 bp, Artificial Sequence
  Parvo primers (Parvovirus B19)
ttctcctctaggttctgcatga

SEQ ID NO: 132 - DNA, 23 bp, Artificial Sequence
  RSV A_B 4 primers (Respiratory syncytial virus)
gagcatactcatacacctccaca

SEQ ID NO: 133 - DNA, 23 bp, Artificial Sequence
  RSV Pr1 (Respiratory syncytial virus)
ctgattttgctaagactccccac

SEQ ID NO: 134 - DNA, 22 bp, Artificial Sequence
  Sal_2 primers (Salmonella typhimurium, strain LT2)
tgataaacttcatcgcaccgtc

SEQ ID NO: 135 - DNA, 20 bp, Artificial Sequence
  TB_3 primers (Mycobacterium tuberculosis)
aggatcctgcgacgtaggcg

SEQ ID NO: 136 - DNA, 25 bp, Artificial Sequence
  Zika Pr1 (Zika virus)
aagttcttcttcacactgccttttc

SEQ ID NO: 137 - DNA, 21 bp, Artificial Sequence
  Zika_2 (Zika virus)
ttatcagtgcgtggaacaacc

SEQ ID NO: 138 - DNA, 18 bp, Artificial Sequence
  Zika Pr3 (Zika virus)
tcagtgcgtggaacaacc

SEQ ID NO: 139 - DNA, 25 bp, Artificial Sequence
  Dengue Pr1 (Dengue virus)
cctaagatttctagccatacctcca

SEQ ID NO: 140 - DNA, 25 bp, Artificial Sequence
  Dengue Pr2 (Dengue virus)
cctaagatttctagccatacctcca

SEQ ID NO: 141 - DNA, 19 bp, Artificial Sequence
  Dev2 primers (artificial)
ttgccgacgacgaaagcga

SEQ ID NO: 142 - DNA, 22 bp, Artificial Sequence
  FluAH3N1_5 primers (Influenza A virus, strain H3N1)
ggatttgtgttcacgctcaccg

SEQ ID NO: 143 - DNA, 21 bp, Artificial Sequence
  FluA_180817_H3N2_2/3 (Influenza A virus, strain H3N2)
agggaagaatatcgaaaggaa

SEQ ID NO: 144 - skipped

SEQ ID NO: 145 - DNA, 25 bp, Artificial Sequence
  HAV primers (Hepatitis A virus)
cggatattggtgagttgttaagaca

SEQ ID NO: 146 - DNA, 20 bp, Artificial Sequence
  HBV primers (Hepatitis B virus)
tttgtcctggttatcgctgg

SEQ ID NO: 147 - DNA, 17 bp, Artificial Sequence
  HCV O Pr1 (Hepatitis C virus)
ttgggtcgcgaaaggcc

SEQ ID NO: 148 - DNA, 17 bp, Artificial Sequence
  HCV Pr4 (Hepatitis C virus)
tggagatttgggcgtgc

SEQ ID NO: 149 - DNA, 19 bp, Artificial Sequence
  HCV Pr5 (Hepatitis C virus)
tgcccccgcaagactgcta

SEQ ID NO: 150 - DNA, 20 bp, Artificial Sequence
  HCV Pr6 (Hepatitis C virus)
cgaaaggccttgtggtactg

SEQ ID NO: 151 - DNA, 17 bp, Artificial Sequence
  HCV 10 Primers (Hepatitis C virus)
ttgggtcgcgaaaggcc

SEQ ID NO: 152 - DNA, 25 bp, Artificial Sequence
  HIV primer 1 (Human immunodeficiency virus-1)
gcaaagatcattagggattatggaa

SEQ ID NO: 153 - DNA, 26 bp, Artificial Sequence
  Malaria primers (All species of plasmodium)
cccttaacgtaaagatcatttatgaa

SEQ ID NO: 154 - DNA, 19 bp, Artificial Sequence
  MS2 primers (Bacteriophage MS2)
tgcaggatgcagcgcctta

SEQ ID NO: 155 - DNA, 20 bp, Artificial Sequence
  Parvo primers (Parvovirus B19)
taactatgttgggcctggca

SEQ ID NO: 156 - DNA, 20 bp, Artificial Sequence
  RSV A_B 4 primers (Respiratory syncytial virus)
tattgggcaatgctgctggc

SEQ ID NO: 157 - DNA, 22 bp, Artificial Sequence
  RSV Pr1 (Respiratory syncytial virus)
ccaaaaattgggtggtgaagca

SEQ ID NO: 158 - DNA, 21 bp, Artificial Sequence
  Sal_2 primers (Salmonella typhimurium, strain LT2)
tatggatttgtcctccgccct

SEQ ID NO: 159 - DNA, 24 bp, Artificial Sequence
  TB_3 primers (Mycobacterium tuberculosis)
gaaggcgtactcgacctgaaagac

SEQ ID NO: 160 - DNA, 19 bp, Artificial Sequence
  Zika Pr1 (Zika virus)
tcacaaggagtgggaagcg

SEQ ID NO: 161 - DNA, 20 bp, Artificial Sequence
  Zika_2 (Zika virus)
ggggggtttatgctcctctc

SEQ ID NO: 162 - DNA, 17 bp, Artificial Sequence
  Zika Pr3 (Zika virus)
gcggggggtttatgctc